SEQUENCE LISTING (2 sequences)

SEQ ID NO: 1 - DNA, 6611 bp, Homo sapiens
taacgagcttccttccaccgcaaaagagctggagaacaatgctaggcaacgtgctggagaccttggccctcggaacccaagtcacgcctcccatgtgagctctggagggagaactttatgtgttgcactgagggcagtctccggaaacgcgattcgcagcgggcgccggaagcggtgttgtgtctgcagctctggcagaggactgttccactagacacgctgaagggactgggtacgtgttttccttcaggaccagagctgagaggagctgggatcgcggcggcaatggaacgggcctcagaaaggcgcacggccagcgcgctttttgcggggttccgggccttgggacttttcagcaacgacattccacacgtggtgcggttcagcgcgctcaagcgccggttctatgtaacaacctgcgtgggcaagagtttccacacctatgacgttcagaaacttagtctggttgcagtaagtaattctgttccacaggatatctgctgtatggcagctgatggcagattagtctttgctgcttatggaaatgttttctctgcatttgcccgtaataaagagatagtacatacctttaagggtcataaggcagaaatccatttcttgcaaccctttggagaccacattatctctgttgatactgatggcattcttattatttggcacatatattcagaagaagaatacctgcagttgacttttgataaatcagtatttaaaatttctgcaattttgcatccaagtacctacttgaataaaatacttctgggcagtgaacaaggaagcctgcagttgtggaatgtaaaatccaataaacttctatatacatttccaggatggaaagttggagtgacagctcttcagcaggcaccagccgtggatgttgttgctattggtcttatgtcaggtcaagttatcattcacaacattaaatttaatgaaacattaatgaagtttcgtcaagactggggacccattacttcaatttcatttcgcacagatggtcatccagtaatggcagctggaagcccatgtggccatattggactctgggatctagaagacaaaaaattaatcaaccaaatgagaaatgcacactctacagcaattgccggactgacatttctccatagagagccacttcttgtcacaaatggcgctgacaatgctcttaggatatggatatttgatggtcctacaggtgaaggccgacttttgagattcagaatgggtcatagtgctcctcttaccaatatcagatattatggacagaatggacagcagattctaagtgcaagtcaagatggaactcttcagtcattttccacggtacatgaaaaattcaataagagcttgggacatggattaataaataaaaagagagttaaacgtaaaggacttcagaataccatgtcagtgagacttccacccatcacaaagtttgcagcagaggaagctcgtgaaagtgactgggatggtatcattgcttgccatcaaggtaagctatcttgctcaacctggaattatcagaaatctacaataggcgcttactttctcaagccaaaagagttgaagaaagatgacataactgcaacagcagtggatataacttcttgtggaaactttgctgtaattggcctctcatcaggaactgtagatgtatataacatgcagtctggcatacatcgaggaagttttggcaaggatcaagctcacaagggatctgttagaggtgtcgcagtggatggattaaaccagttgacagttacaactggtagtgaaggattactcaaattctggaactttaaaaacaaaattttaatccattctgtgagcctcagttcatctccaaatatcatgttgctacatagggacagtggcattctgggactcgccttggatgacttctccattagtgttctggacatagaaactaggaagattgtcagagagttttctggacaccaaggccaaataaatgacatggcttttagtcctgatggtcgttggttaataagtgctgcgatggattgctctattaggacttgggaccttccttctgggtgccttatagactgctttttgttggactcggctcctctcaatgtttctatgtctcctactggagactttctggcaacttcccatgtggaccaccttggaatttatctatggtccaatatttccctgtattcagttgtttcattacggccacttcctgcagattatgtcccttcaatagtcatgcttcctggtacttgtcaaacccaagatgtagaagtatcagaagaaacagtagaaccaagtgatgaattgatagaatatgattcgccagaacagttgaatgagcaattggtgactctttcacttcttcctgaatcacgatggaaaaaccttcttaaccttgatgttattaagaaaaagaataaaccaaaggaaccacccaaagtacccaaatcagcaccatttttcattccaacaattcctggccttgtacccagatatgctgcacctgaacaaaataatgatccccagcagtctaaagtggtaaatcttggagttttggctcaaaaatcagatttctgcttgaaacttgaagaaggactggtaaataataagtatgacactgctctcaaccttctgaaagaatcaggcccatcaggaattgaaacagagctgcgaagcttgtctcctgattgtggtgggtccatagaagttatgcagagcttcttgaaaatgattgggatgatgctggacagaaagcgtgattttgagttagcccaggcataccttgcattgtttctaaagttacaccttaaaatgcttccttcagagccagtactcctagaagaaataacaaatttgtcatcccaggtggaagaaaactggacccatttgcaatcactcttcaatcaaagcatgtgtattttaaattatctcaaaagtgctttgttgtaaaaataaatttgtgactaaacaaagactttcatattaaatgggttcaattgaactcatttcttattttccaagtgtcaatgtgaaaagaaaataaatgctagcactactgactagtcagtatatctccactttaaatgctaaatactttcttgaaataaaatcgcacctcccggccaggcatgatggataatgcctgtaattccagcactttcagaggccaaagcgggaggattgcttgaagccaggaatttgagcccagcctgggcaacatagcaagcaagatcccatctctacaaaaagttaaaaaaataaattacacctgccttttgttttgaagacttaaagaatctgctcaaatgatttattttctgagtctcttcacattaacttttatatgatttttaaaaaattattacctgcttatattttttgagtatctgtttcatataaagagatgctgcatgttcactgtgaagtattgctgaattaaattgaaaatgtcagataaaactgatacatgatgtagttataaaaatgctgttatgaagaaaaaaggaatgcttttttcctcggtggcctttgataaaaatatgaatgattgactctcagagttggaggataaactaatcatcttctagcacagccctgttgtgttgcaggagaggaatcaggctaaagtctgctggtatttcagtgacagattaaaactttctaacagtggcctagtgctttctccattgtaatgtactgaaaggaagttgttcatggatcagaaatgtgggaggcccttccttgcccaaggcaagtcgctgtagatggttaaactgataccaaggataggtaagggaaattccaaattccattggaagatggccttttactgacactgcagacatgtagataacaatagtttaatgtcctctgaaaaggtaaaaattccataaatccttgcaaacaatttttagcaatattgtttattgctaccacagccaagaatgggtatttcaaagccagggttttttttattttgttttgttttgtttttttttttttttttttggctacacttttttgggggggaccttttaatttgggaagaaaatgctgagtatacttttctttcacaaccattgcaaacccaggtagccctggagttttctttttttctcatcatcattgttttgttttttttatggttcaagacttttgcaatacatgaattaaaaataagcagttctttaaagcatagcgctctaaaaactattcttagtttataccctcaaaagttgggtctttcatgaaacacagtggtttgaatatgaacaccagtagcattctcgttcgttgttagggtaagagagttttgtagaatgatctccaaaagaagcagtcctcaaaaggcatttaccagtgatatcttttaatattcatgtttttatttaatgatgtaagatagaattaataaactactaaggaaataagccaatgtggttattgtactcaatttcgtttttcttttagaaatgtgtattgacttattaaagccactgacagaaatgttaatcatgacttaagttctaatttaaaaaactattttcctctttcctaaatactatattctaaattggaatatggcaaaatcccaatatataattctatacctagtaaagattgtaaataactaaaacaaaatgataagagctgtgaactgaaattgtattgcttacagaaatcatgaaccgaattttcttctctttttaaagtgagcataaagacttaagtgtattgttaccagagatatatttagatagaatacatcattttggcaggtaatcttctagaacctttcatgtactgtttccatcaagggagggaaagaaatagaaaatataattttatctatattttccttaaaatatatcaatctgtgcacactctttcagttttttaattactcatgtaatatatgactacattttgcttgtgtgctttttgaaaaccatcttcagtcccaatatcctctgtagaagtaatagttttaactctcctagatcttcttttagagtgtttttatgagtgtttaatgtaggaatgtgtatatattatctacagccacaggaaatgtatagttacgatttttacatacgtgtatgtatattattgtacaacttgctatttctacttgggagttttttattttggtacattcacatttacctaatctcttgtgcactgtccaacagtactttgcctataatagacacttaaggaagttttccttctttttattcctttataattacaaacaatgctgaaaggaacatcatcctacatgagccctttttgtacatataaggctctctctgtggtatatgcttaggatttgaatttcaggccagagtataaccatttttaatttgaagagatacttccatttgggttccagaatggctgtatcaagttacagttcccctgagcaagatatgagcatctgcttccccatgtaattaatgacacttgctattggatttttgtaatttaattactctgaaaaaattattttggttttaaatcagaaatttctcactagtgaagatggacatatttttgtatgtttattttagaacatataaacatttgttaattttctttttcctctcatttttctattgaatgatttgtccttttatctcatggatttgttaggagttctcttgtattttctgtattttatgtctgttgccaatgttttctcctacttagccatttggctttaattttacatggtgtctgtttaaataaatgttttatatttgtatacaatcaaattgattaattttcatgacttatttcaggatataaagcaaattattttctaagacatttacagtgtatgttttcctatgtttacatgtgctcatttcacgtcatgtattttcctctacgacatgtttctgtgtgagtagttaacatgtagatctttagaattgatttttctgaagagtgaaacagcgctgcttccaatatctgttcaaaattcatttattgaaaagtccactcatatgtctgatttgaaatgctgtttcattatgtgttaaattctttcactgagctcttccatattcctagtaaatgatgcattactttggtatgttctcagatttgggtaaagccttcctcatgttctataaaaattggttaactgctggtgagcacatctcttgaagttttgatatgggttgcacaacatttataggactgatttggagagaccgacaataccgagtgcagtcctcccttggtatctagttccaggacccctgtggataccaaaatccaagcaatgccctgcagatcatgggacctatgcatatgaaaagtcggtccttgtatttgagggttttgcatccctcaaataccatatgttttatttgtgtttggttgaaaaaagtttgcatataagtgaaacttagcatttcaaacctgtgttgttcaaagatcatttgtatttctcttcaagaacaatgaatgtcttcattaaatcatcttttgtgggagagcttttggtttcatagattttcatatatatatatgatataataaacactttttgacattcaaaaaaaaaaaaaaaaaaa

SEQ ID NO: 2 - protein, 951 aa, Homo sapiens
MCCTEGSLRKRDSQRAPEAVLCLQLWQRTVPLDTLKGLGTCFPSGPELRGAGIAAAMERASERRTASALFAGFRALGLFSNDIPHVVRFSALKRRFYVTTCVGKSFHTYDVQKLSLVAVSNSVPQDICCMAADGRLVFAAYGNVFSAFARNKEIVHTFKGHKAEIHFLQPFGDHIISVDTDGILIIWHIYSEEEYLQLTFDKSVFKISAILHPSTYLNKILLGSEQGSLQLWNVKSNKLLYTFPGWKVGVTALQQAPAVDVVAIGLMSGQVIIHNIKFNETLMKFRQDWGPITSISFRTDGHPVMAAGSPCGHIGLWDLEDKKLINQMRNAHSTAIAGLTFLHREPLLVTNGADNALRIWIFDGPTGEGRLLRFRMGHSAPLTNIRYYGQNGQQILSASQDGTLQSFSTVHEKFNKSLGHGLINKKRVKRKGLQNTMSVRLPPITKFAAEEARESDWDGIIACHQGKLSCSTWNYQKSTIGAYFLKPKELKKDDITATAVDITSCGNFAVIGLSSGTVDVYNMQSGIHRGSFGKDQAHKGSVRGVAVDGLNQLTVTTGSEGLLKFWNFKNKILIHSVSLSSSPNIMLLHRDSGILGLALDDFSISVLDIETRKIVREFSGHQGQINDMAFSPDGRWLISAAMDCSIRTWDLPSGCLIDCFLLDSAPLNVSMSPTGDFLATSHVDHLGIYLWSNISLYSVVSLRPLPADYVPSIVMLPGTCQTQDVEVSEETVEPSDELIEYDSPEQLNEQLVTLSLLPESRWKNLLNLDVIKKKNKPKEPPKVPKSAPFFIPTIPGLVPRYAAPEQNNDPQQSKVVNLGVLAQKSDFCLKLEEGLVNNKYDTALNLLKESGPSGIETELRSLSPDCGGSIEVMQSFLKMIGMMLDRKRDFELAQAYLALFLKLHLKMLPSEPVLLEEITNLSSQVEENWTHLQSLFNQSMCILNYLKSALL

(19) United States
(12) Patent Application Publication
(10) Pub. No.: US 2007/0172919 A1
(43) Pub. Date: Jul. 26, 2007

(54) WDR36 Gene Alterations and Glaucoma

(75) Inventors: Mansoor Sarfarazi, Farmington, CT (US); Sharareh Monemi, Southbury, CT (US)

Correspondence Address: CANTOR COLBURN, LLP, 55 GRIFFIN ROAD SOUTH, BLOOMFIELD, CT, 06002, US

(73) Assignee: THE UNIVERSITY OF CONNECTICUT, Farmington, CT (US)

(21) Appl. No.: 11/627,658

(22) Filed: Jan. 26, 2007

Related U.S. Application Data
(60) Provisional application No. 60/762,556, filed on Jan. 26, 2006.

(51) Int. Cl. C07K 14/705; C07H 21/04; C12P 21/06

(52) U.S. Cl. 435/69.1; 435/320.1; 435/325; 530/350; 536/23.5

(57) ABSTRACT

Disclosed herein is the mapping of a new adult-onset primary open-angle glaucoma (POAG) locus on 5q22.1 (GLC1G) and identification of its defective gene. Mutation screening of 7 candidate genes from the GLC1G critical region identified the WDR36 (WD40-Repeat 36) gene. Methods of detection, prognosis and diagnosis of the presence or absence of glaucoma or of an increased risk of glaucoma are described, in which a sample is tested for the presence of glaucoma-causing alterations or glaucoma-susceptibility alteration in the GLC1G locus on chromosome band 5q22.1, such as in the WDR36 gene.

### RELATED APPLICATIONS

This application claims the benefit of U.S. Provisional Application No.: 60/762,556, filed on Jan. 26, 2006, the contents of which are incorporated herein by reference in their entirety.

### GOVERNMENT SUPPORT

The invention was supported, in whole or in part, by grant RO1-EY09947 from the National Institutes of Health (National Eye Institute). The United States Government has certain rights in the invention.

### BACKGROUND OF THE INVENTION

Glaucoma is a group of ocular disorders characterized by a specific pattern of optic nerve and visual field defects. This condition is one of the two leading causes of blindness, affecting over 67 million people worldwide. Open-angle glaucoma is usually asymptomatic until the late stages of the disease, by which time significant and irreversible optic nerve damage has already taken place. As the sensitivity of current diagnostic techniques is suboptimal, the diagnosis of glaucoma is usually made once an irreversible damage has already occurred. As glaucoma related visual loss is preventable in many cases, there is an urgent need to diagnose glaucoma at its early stages and to institute appropriate neuroprotective management of the ganglion cells. Mapping, cloning and identification of novel mutations involved in the etiology of glaucoma provide a significant opportunity for presymptomatic diagnosis, improved prognosis, and better understanding of the etiology of this blinding condition.

Adult-onset primary open-angle glaucoma (POAG; MIM#137760) is the most common form of this ocular group, usually manifesting itself after the age of 40 years. The prevalence of POAG is about 1-2% over age 50 in white populations, and 4-5 times greater in black populations of the same age. Clinical diagnosis of all groups is based on characteristic changes of the optic nerve head and visual field, which are usually accompanied by increased intraocular pressure (IOP). Family history is an important risk factor for POAG and genetic study of families shows that dominant genes are predominantly involved in this condition.

During the last decade, a total of 17 genetic loci and two genes have been reported for POAG: Myocilin (MYOC) is primarily mutated in juvenile-onset subjects while Optineurin (OPTN) is mainly mutated in normal-pressure POAG individuals

Disclosed herein is the identification of a new POAG locus and the use of this locus to determine the presence or absence of glaucoma or the risk of glaucoma in human patients.

### SUMMARY OF THE INVENTION

In one embodiment, an isolated nucleic acid molecule comprises a portion of a GLC1G locus on chromosome band 5q22.1, wherein the nucleic acid has an alteration in a nucleotide sequence, and wherein the alteration is a glaucoma-causing alteration or a glaucoma-susceptibility alteration. In another embodiment, the alteration is located in the WDR36 gene. Also included is an isolated or purified polypeptide encoded by the nucleic acid molecule.

In one embodiment, a method for detecting the presence or absence of a WDR36-associated glaucoma or of a WDR36-associated greater than normal risk of glaucoma in an individual comprises assessing a sample from a patient for an alteration in a WDR36-associated nucleic acid, or assessing the sample for an alteration in a WDR36-associated polypeptide, wherein the alteration in the nucleic acid or in the polypeptide is a glaucoma-causing alteration or a glaucoma-susceptibility alteration.

### BRIEF DESCRIPTION OF THE DRAWINGS

FIG. 1 depicts a partial genetic and physical map of chromosome 5q. Cytogenetic banding and map positions (in Megabases) for a selected group of DNA markers are shown. The order of 7 known genes in this overlapping region that were fully sequenced in our study are also shown. D5S2501 maps between AK125070 and BC017169.

FIG. 2 depicts the predicted POAG-causing mutations in the WDR36 gene. a-b. Partial reverse sequence for N355S (AAT>AGT) and A449T (GCA>ACA). C-d. Partial forvard electropherogram for R529Q (CGA>CAA) and D658G (GAC>GGC). The vertical line identifies the intron/exon boundaries for A449T and D658G sequences. e. Segregation of D658G mutation in our original GLC1G-linked family of POAG-527. Bgl I restriction digestion of a 389-bp PCR fragment produces 2 products of 272-bp and 117-bp only in presence of the mutant allele. For simplicity, only 7 affected (filled-in), 9 gene carriers (dot inside their symbols), 2 normal subjects and 4 available spouses are shown. All other normal subjects with no D658G mutation in the 3^{rd} and 4^{th} generations were omitted.

FIG. 3 shows the partial amino acid alignment and evolutionary conservation of 12 WDR36 variations in human and 4 other species.

FIG. 4 shows WDR36 gene expression in various human tissues. a. Northern blotting of non-ocular tissues identified two transcripts of approximately 5.9-kb and 2.5-kb (top panel). Beta-Actin was used as a standard control b. mRNA expression patterns of WDR36 in various non-ocular tissues by RT-PCR. c. RT-PCR of WDR36 in a group of human ocular tissues.

FIG. 5 shows Wdr36 gene expression in mouse. a. Northern blot analysis identified two transcripts of approximately 3.5-kb and 2.9-kb. b. mRNA gene expression in various adult mouse tissues. c. RT-PCR of Wdr36 in mouse embryo at different gestational ages. The earliest gene expression was at embryonic day 7.

The foregoing and other objects, features and advantages of the invention will be apparent from the following more particular description of preferred embodiments of the invention, as illustrated in the accompanying drawings.

### DETAILED DESCRIPTION OF THE INVENTION

The invention relates generally to the mapping and identification of novel gene alterations (e.g., mutations) involved in the etiology of glaucoma. The invention includes a new, previously unknown genetic locus for POAG on chromosome band 5q22.1. This locus has been designated as GLC1G. The invention also includes a gene in the GLC1G locus, the WDR36 (WD40-repeat 36) gene, as a novel causative gene for POAG. The invention farther includes the demonstration that the WDR36 gene is expressed in a number of ocular tissues as well as in non-ocular tissues.

More specifically, certain alterations of the WDR36 gene have been identified that are present in a population of POAG subjects, but absent in normal subjects unaffected by glaucoma. The gene alterations (e.g., mutations) identified herein are found in POAG subjects having elevated intraocular pressure (IOP) and in POAG subjects with normal levels of IOP. Certain gene variants are predicted glaucoma (e.g., POAG)-causing alterations and other gene variants are glaucoma (e.g., POAG)-susceptibility alterations. Such alterations are associated with the presence of glaucoma and/or with a greater than normal risk of glaucoma. Included are alterations in the amino acid sequences of the protein encoded by the WDR36 gene, wherein the alterations are glaucoma-causing alterations or glaucoma-susceptibility alterations. Disclosed herein are methods of detecting the presence or absence of alterations in the WDR36 gene and/or polypeptide that are glaucoma-causing mutations or potential glaucoma-susceptibility mutations. Alterations in amino acid sequence corresponding to predicted disease-causing mutations, most of which alter the amino acid sequence of one or another of the about eight WD40 repeats in the G-beta subunit of WDR36 protein, are included. Mutations in the WD40 repeats can alter protein structure, and thereby alter interaction of WDR36 with other proteins.

Included herein is the detection of alterations in the GLC1G locus (e.g., alterations in the WDR36 locus), wherein the alterations are glaucoma-causing alterations or glaucoma-susceptibility alterations. Such alterations are associated with the presence of glaucoma and/or with a greater than normal risk of glaucoma. Also included are methods for the prognosis, i.e., pre-symptomatic detection, and diagnosis of glaucoma. More particularly, the detection of individuals with primary open-angle glaucoma (POAG) or with a risk for developing POAG by detecting alterations, i.e., variants or mutations in a gene associated with POAG, such as the WDR36 gene, is included. Detection of POAG or of a risk of POAG by detecting variants of the WDR36 gene includes detection of adult-onset POAG and other forms of open-angle glaucoma, such as juvenile-onset open-angle glaucoma. The alterations may be in a coding sequence of, for example, the WDR36 gene, or in non-coding sequences such as promoter regions, 5′ untranslated regions, 3′ untranslated regions, and introns. The alterations may be changes of one to a few amino acids of a protein or nucleotides of a nucleic acid, or larger DNA alterations and/or rearrangements, such as deletions, insertions and duplications.

In another embodiment, included are methods for the prognosis and diagnosis of glaucoma by the detection of alterations in the amino acid sequence of one or more structurally important WD40 repeats and/or other functionally important domains of the WDR36 protein. More specifically, the glaucoma detected is open-angle glaucoma, such as POAG including adult-onset POAG.

Described herein is the mapping of a new locus for POAG (GLC1G) on the 5q22.1 chromosome band. The invention also includes the identification of the glaucoma-causing gene on GLC1G: WDR36 (SEQ ID NO:1; GenBank Accession #: NM_{—}139281). As WDR36 is a novel gene, by Northern blot analysis the transcript size of this gene was determined using two commercially available human and mouse mRNA blots and, by RT-PCR on RNA samples that were directly extracted from the isolated ocular tissues. Expression of this gene was established in human ocular and non-ocular as well as in embryonic and adult mouse tissues.

The WDR36 (WD-Repeat protein 36) has recently been identified as one of the genes that is uniquely involved in T cell activation and highly co-regulated with interleukin 2 (IL2). The WDR36 gene encodes for a T-cell activation protein with a minimum of eight WD40 repeats and, therefore, it is also recognized as T-cell activation WD repeat protein (TA-WDRP). The WDR36 (GenBank accession no. NM_{—}139281) transcript is 6,592-bp long, contains 23 exons and encodes for 951 amino acids. As of yet, no differentially spliced forms of this gene have been reported, though one cDNA clone (AL832494) in the public domain is missing the two exons of 2 and 21, which is anticipated to encode for a putative protein that is only 383 amino acids long. The WDR36 encoded protein consists of at least seven known motifs: a Guanine nucleotide binding protein or (G)-beta WD-40 repeat; an AMP-dependent synthesize and ligase motif; a Mini-chromosome maintenance-5 motif (MCM-5); a Cytochrome cd1-nitrite reductase-like motif (C-terminal heme d1); a lethal giant larvae homologue 2 motif (LLGL2); an Utp21-specific WD40 associated putative domain; a Quinoprotein amine dehydrogenase motif; and beta chain-like motif (Qamine_DH_B_like). WDR36 also has been identified as one of the proteins that is uniquely involved in T-cell activation and it is reported to be highly co-regulated with interleukin 2 (IL2). This gene encodes for a T-cell activation protein with a minimum of eight WD40 repeats and, therefore, it is also recognized as T-cell activation wD repeat protein (TA-WDRP).

Accordingly, included herein are methods of therapy for glaucoma, and also methods and kits for determining the presence or absence of glaucoma or of an increased risk of glaucoma in an individual, by detecting the presence or absence of alterations in the WDR36 gene or the WDR36 polypeptide, or by detecting an alteration in activity of the WDR36 polypeptide or of an WDR36-interacting polypeptide. Glaucoma that is associated with the presence of one or more alterations in the WDR36 gene or in the WDR36 polypeptide is referred to herein as “WDR36-associated glaucoma”, and an increased risk of glaucoma associated with one or more alterations in the WDR36 gene or in the WDR36 polypeptide is referred to herein as “WDR36-associated increased risk of glaucoma”.

The term “glaucoma”, as used herein, includes all types of inheritable, familial and sporadic forms of glaucomas, such as primary congenital or infantile glaucoma; primary open angle glaucoma (POAG), including both juvenile-onset and adult- or late-onset POAG; secondary glaucomas; Pseudoexfoliation glaucoma; pigmentary glaucoma; and low tension glaucoma (LTG) (also known as normal tension glaucoma (NTG)/normal pressure glaucoma (NPG)). In particular embodiments, the glaucoma is primary open angle glaucoma (POAG) or the low tension subgroup of POAG. To the extent that the WDR36 gene is implicated, other types of glaucoma are included in this definition including glaucomas occurring in patients with sporadically occurring glaucoma. An “increased risk” of glaucoma, as used herein refers to a likelihood of an individual to develop glaucoma, that is, greater by an amount that is significant, than the likelihood of another individual or population of individuals for developing glaucoma. The methods disclosed herein can be used for detection, including screening, prognosis and diagnosis, of at-risk individuals and/or populations for glaucoma or the risk of glaucoma.

The term “detection” encompasses methods of screening, diagnosis and prognosis of glaucoma. The term “screening” refers to identification of the presence or absence of alterations in a WDR36 gene or polypeptide, wherein the alterations are glaucoma-causing alterations or glaucoma-susceptibility alterations. The term “diagnosis” includes determining that a patient is affected with glaucoma by analyzing the signs and symptoms of the disease. In addition to identification of the presence of an alteration in a WDR36 gene or polypeptide, which is associated with glaucoma, a diagnosis of glaucoma may further include analysis of the family history of the patient and determination of clinical symptoms of glaucoma in the patient. The term “prognosis” refers to predicting a patient's future risk of developing glaucoma, and the future course of the disease. A patient with an alteration in a WDR36 gene or polypeptide, which is associated with glaucoma may be at higher risk for developing glaucoma in the future than a patient with no such mutations.

The term “treatment” refers to ameliorating the symptoms of glaucoma, preventing or delaying the onset of glaucoma, and/or lessening the severity and/or frequency of symptoms associated with glaucoma. Treatment of glaucoma thus refers to treatment after the appearance of the symptoms of glaucoma as well as prophylactic treatment.

In one embodiment, include herein is detection of alterations in the GLC1G locus, wherein the alterations are glaucoma-causing alterations or glaucoma-susceptibility alterations. Such alterations are associated with the presence of glaucoma and/or with a greater than normal risk of glaucoma. Detection (e.g., screening, prognosis or diagnosis) of WDR36-associated glaucoma, or of an WDR36-associated increased risk of glaucoma, can be made by detecting the presence or absence of an alteration in the WDR36 gene that is associated with glaucoma or with an increased risk of glaucoma. As used herein, the term, “WDR36 gene” refers to a nucleic acid (e.g., DNA, RNA, CDNA) encoding a WDR36 polypeptide. A “gene” as used herein comprises not only translated nucleic acids, but also untranslated nucleic acids (e.g., promoter regions, 5′ untranslated regions, 3′ untranslated regions, introns, etc.).

For sequence information, see GenBank Accession#: NM_{—}139281; SEQ ID NO: 1.

The term “isolated nucleic acid molecule” or “purified nucleic acid molecule” includes nucleic acid molecules that are separated from other nucleic acid molecules present in the natural source of the nucleic acid. For example, with regard to genomic DNA, the term “isolated” includes nucleic acid molecules that are separated from the chromosomal sequences with which the genomic DNA is naturally associated. Preferably, an “isolated” nucleic acid is free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ contiguous nucleotide sequences which naturally flank the nucleic acid molecule in genomic DNA from which the nucleic acid is derived. Moreover, an “isolated” nucleic acid molecule, such as a CDNA molecule, can be substantially free of other cellular material, or culture medium when produced by recombinant techniques, or substantially free of chemical precursors or other chemicals when chemically synthesized. By free of other cellular material, it is meant that an isolated nucleic acid molecule is greater than or equal to about 70%, 75%, 80%, 85%, 90%, 95% or 99% pure.

A WDR36 nucleic acid can be produced by recombinant DNA technology using techniques well known in the art. Methods which are well known to those skilled in the art can be used to construct expression vectors containing WDR36 coding sequences and/or antigenic molecule coding sequences and appropriate transcriptional/translational control signals. The coding sequence for WDR36 is operatively linked to the regulatory elements necessary for expression of the nucleic acid molecule. “Operatively linked” refers to a juxtaposition wherein the components so described are in a relationship permitting them to function in their intended manner. An expression control sequence operatively linked to a coding sequence is ligated such that expression of the coding sequence is achieved under conditions compatible within the expression control sequences. As used herein, the term “expression control sequences” refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signals for introns (if introns are present), maintenance of the correct reading frame of that gene to permit proper translation of the mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter. By “promoter” is meant a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included. Suitable methods for recombinant expression include, for example, in vitro recombinant DNA techniques, synthetic techniques and in vivo recombination/genetic recombination.

A variety of host-expression vector systems can be utilized to express the WDR36 gene. These include but are not limited to microorganisms such as bacteria (e.g., E. coli, Bacillus subtilis) transformed with recombinant bacteriophage DNA, plasmid DNA or cosmid DNA expression vectors containing the WDR36 coding sequence; yeast (e.g., Saccharomyces, Pichia) transformed with recombinant yeast expression vectors containing the WDR36 coding sequence; insect cell systems infected with recombinant virus expression vectors (e. g., baculovirus) containing the WDR36 coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the WDR36 coding sequence; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, for example, vectors which direct the expression of high levels of fusion protein products that are readily purified may be desirable. Such vectors include, but are not limited, to the E. coli expression vector pUR278 (accession number L09146), in which the WDR36 coding sequence can be ligated individually into the vector in frame with the lac Z coding region so that a fusion protein is produced; pIN vectors; and the like. pGEX vectors can also be used to express foreign polypeptides as fusion proteins with glutathione S-transferase (GST). In general, such fusion proteins are soluble and can easily be purified from used cells by adsorption to glutathione-agarose beads followed by elution in the presence of free glutathione. The pGEX vectors are designed to include thrombin or factor Xa protease cleavage sites so that the cloned WDR36 gene protein can be released from the GST moiety.

In one embodiment, an insect system, Autographa californica nuclear polyhedrosis virus (AcNPV) is used as a vector to express foreign genes. The virus grows in Spodoptera frugiperda cells. The WDR36 gene can be cloned individually into non-essential regions (for example the polyhedrin gene) of the virus and placed under control of an AcNPV promoter (for example the polyhedrin promoter). Successful insertion of the WDR36 coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed.

In mammalian host cells, a number of viral-based expression systems may be utilized. In cases where an adenovirus is used as an expression vector, the WDR36 coding sequence can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing WDR36 in infected hosts. Specific initiation signals may also be required for efficient translation of inserted WDR36 coding sequence. These signals include the ATG initiation codon and adjacent sequences. The efficiency of expression can be enhanced by the inclusion of appropriate transcription enhancer elements, transcription terminators, etc.).

In general, any type of mammalian expression vector can be used, although those with the highest transfection and expression efficiencies are preferred to maximize the levels of expression. Specific types of vectors which can be employed include herpes simplex viral based vectors such as pHSV1; recombinant retroviral vectors such as MFG Moloney-based retroviral vectors including LN, LNSX, LNCX, and LXSN; vaccinia viral vectors including KVA; recombinant adenovirus vectors such as pJM17; second generation adenovirus vectors such as DE1/DE4 adenoviral vectors; and Adeno-associated viral vectors such as AAV/Neo.

In addition, a host cell strain can be chosen which modulates the expression of the inserted sequences, or modifies and processes the WDR36 in the specific fashion desired. Different host cells have characteristic and specific mechanisms for the post-translational processing and modification of proteins such as glycosylation. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be employed. Such mammalian host cells include but are not limited to CHO, VERO, BHK, HeLa, COS, MDCK, 293, 3T3, W138, etc.

An “alteration” is a change (e.g., insertion, deletion, or change in one or more nucleotides) of the nucleic acid encoding WDR36 polypeptide, as compared with the known sequence of nucleic acid encoding WDR36 (SEQ ID NO:1). Alterations include mutations in the WDR36 gene, such as the insertion or deletion of a single nucleotide, or of more than one nucleotide, resulting in a frame shift mutation; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of the gene; duplication of all or a part of the gene; transposition of all or a part of the gene; or rearrangement of all or a part of the gene. More than one such mutation may be present in a single gene. Such sequence changes (e.g., exon shuffling) cause a mutation in the polypeptide encoded by the WDR36 gene. For example, if the mutation is a frame shift mutation, the frame shift can result in a change in the encoded amino acids, and/or can result in the generation of a premature stop codon, causing generation of a truncated polypeptide. Alternatively, an alteration associated with glaucoma can be a sequence alteration that does not result in a change in the polypeptide encoded by the WDR36 gene. Such an alteration may alter splicing sites, affect the stability or transport of mRNA, or otherwise affect the transcription or translation of the gene. A WDR36 gene that has any of the mutations or sequence alterations described above is referred to herein as a “mutant gene”.

A polymorphism is a DNA sequence variation occurring when a single location in the genome (or other shared sequence) differs between members of a species (or between paired chromosomes in an individual). A single nucleotide polymorphism (SNP) is a single nucleotide change in a sequence. SNPs may fall within coding sequences of genes, noncoding regions of genes, or in the intergenic regions between genes. Variations in the DNA sequences of humans can affect how humans develop disorders such as glaucoma. A convenient method for detecting SNPs is restriction fragment length polymorphism (SNP-RFLP). If one allele contains a recognition site for a restriction enzyme while the other does not, digestion of the two alleles will give rise to fragments of different length. Currently, existing SNPs are most easily studied using microarrays. Microarrays allow the simultaneous testing of up to hundreds of thousands of separate SNPs and are quickly screened by computer.

Alterations in the WDR36 gene include predicted disease-causing mutations, potential disease-susceptibility mutations, amino acid polymorphisms, intronic polymorphisms, or a combination comprising one or more of the foregoing alterations. In one embodiment, a predicted disease-causing mutation occurs in a WD40 domain of the WDR36 protein, such as, for example, the 4^{th}, 6^{th}, or 8^{th} WD40 repeat, or a combination thereof.

In specific embodiments of the invention, the alteration is a predicted disease-causing mutation including a change of 91C>A in the WDR36 gene, corresponding to an P to T change at codon 31 (P31T); a change of 1064A>G in the WDR36 gene, corresponding to an N to S change at codon 355 (N355S); a change of 1345G>A in the WDR36 gene, corresponding to an A to T change at codon 449 (A449T); a change of 1514G>T in the WDR36 gene, corresponding to a C to F change at codon 505 (C505F); a change of 1586G>A in the WDR36 gene, corresponding to an R to Q change at codon 529 (R529Q); a change of 1642G>A in the WDR36 gene, corresponding to a D to N change at codon 548 (D548N); a change of 1973A>G in the WDR36 gene, corresponding to an D to G change at codon 658 (D658G), or a combination comprising one or more of the foregoing alterations. In other embodiments, the change is a potential disease-susceptibility mutation, including a change of 74T>C in the WDR36 gene, corresponding to an L to P change at codon 25 (L25P); a change of 488C>T in the WDR36 gene, corresponding to an A to V change at codon 163 (A163V); a change of 646-647TA>CC in the WDR36 gene, corresponding to a H to P change at codon 212 (H212P); or a combination comprising one or more of the foregoing alterations.

In specific embodiments of the invention, the alteration is an amino acid polymorphisms including a change of 402C>T in the WDR36 gene, corresponding to G134G; a change of 790A>G, corresponding to 1264V; a change of 2011A>G, corresponding to M671V; a change of 0.2142C>G, corresponding to V714V; a change of 2181A>T, corresponding to V727V; or a combination comprising one or more of the foregoing alterations.

In other specific embodiments, the alteration is an intronic polymorphism including IVS3−113G>A; IVS4−27A>G; IVS4−139A>T; IVS5+30C>T; IVS7+105A>G; IVST7−39T>G; IVS8+92G>A; IVS 12+90C>T; IVS 13+89G>A; IVS 14+89C>A; IVS16−30A>G; IVS18−83A>G; IVS18+216C>T; IVS21+60G>C; IVS22+129G>A; IVS22+189T>C; IVS22−202A>G; 3′ UTR+45C>G; or a combination comprising one or more of the foregoing alterations.

A method of detecting an indication (e.g., the presence or absence) of a WDR36-associated glaucoma or a WDR36-associated risk of glaucoma in a sample from an individual comprises assessing the sample for an alteration in a WDR36 nucleic acid or an alteration in an WDR36 polypeptide, wherein the alterations are glaucoma-causing alterations or glaucoma-susceptibility alterations. Such alterations are associated with the presence of glaucoma and/or with a greater than normal risk of glaucoma. Detection of alterations in a WDR36 nucleic acid or polypeptide can be used to screen individuals for a WDR36-associated glaucoma or a WDR36-associated risk of glaucoma. In specific embodiments, the alterations are the aforementioned predicted disease-causing mutations, potential disease-susceptibility mutations, amino acid polymorphisms, intronic polymorphisms, or a combination comprising one or more of the foregoing alterations.

In a first method of detection of glaucoma or an increased risk of glaucoma, hybridization methods. For example, a test sample of genomic DNA, RNA, or cDNA, is obtained from an individual, such as, for example, an individual suspected of having, carrying a defect for, or being at increased risk for, glaucoma (the “test individual”). Suitable individuals include an adult, child, or fetus. Suitable test samples are from a sources which contains the nucleic acid (e.g., DNA, RNA), such as a blood sample, serum sample, lymph sample, sample of fluid from the eye (e.g., fluid from the anterior chamber), sample of amniotic fluid, sample of cerebrospinal fluid, or tissue sample from skin, muscle, buccal or conjunctival mucosa, placenta, gastrointestinal tract, or other organs. A test sample of DNA from fetal cells or tissue can be obtained by appropriate methods, such as by amniocentesis or chorionic villus sampling. The DNA, RNA, or cDNA sample is then examined to determine whether an alteration in the WDR36 gene is present.

If desired, amplification of the sample (e.g., by polymerase chain reaction) is performed prior to assessment for the presence or absence of the alteration in the WDR36 gene. Amplification is employed for all, or a portion of the nucleic acid comprising the WDR36 gene so long as the portion contains the part of the WDR36 gene that comprises the alteration (e.g., one or more exons, such as exon 8, exon 11, exon 13, exon 17, or other exons comprising an alteration, as described below). In one embodiment, a portion comprises at least one exon of the WDR36 gene.

In one embodiment, the presence or absence of the alteration is indicated by hybridization of the gene in the genomic DNA, RNA, or cDNA to a nucleic acid probe. A “nucleic acid probe”, as used herein, is a single-stranded polynucleotide which hybridizes to the gene of interest (WDR36). The probe comprises at least a portion of contiguous sequence of SEQ ID NO: 1, or a complement thereof. A nucleic acid probe can be an oligonucleotide of about 10 to about 100 contiguous nucleotides of WDR36 or an alteration thereof, specifically about 15 to about 60 nucleotides, or a piece of nucleic acid which may be several hundred kb long. Short probes generally require cooler temperatures to form sufficiently stable hybrid complexes with the template. Suitable nucleic acid probes include a DNA probe or an RNA probe, wherein the nucleic acid probe comprises at least one alteration in the WDR36 gene. The probe comprises the entire gene, a gene fragment, a vector comprising the gene, an exon of the gene, or a portion of nucleic acid which may be several hundred kilobases long, and the like. Preferred nucleic acid probes are those comprising an alteration in the WDR36 gene.

In another embodiment, allele-specific oligonucleotides are used to detect the presence of an alteration in the WDR36 gene. An “allele-specific oligonucleotide” (also referred to herein as an “allele-specific oligonucleotide probe”) is an oligonucleotide of approximately 10-100 contiguous bases, specifically approximately 15-60 contiguous bases of the WDR36 sequence, that specifically hybridizes to the WDR36 gene, and that contains an alteration associated with glaucoma or with increased risk of glaucoma. An allele-specific oligonucleotide probe that is specific for particular alterations in the WDR36 gene can be prepared, using standard methods. To identify alterations in the gene that are associated with glaucoma or with an increased risk of glaucoma, a test sample of DNA is obtained from the individual. PCR can be used to amplify all or a fragment of the WDR36 gene, and its flanking sequences. The DNA containing the amplified WDR36 gene (or fragment of the gene) is then hybridized to the allele-specific probe. Specific hybridization of an allele-specific oligonucleotide probe to DNA from the individual is indicative of an alteration in the WDR36 gene that is a glaucoma-causing alteration or a glaucoma-susceptibility alteration. An absence of specific hybridization of an allele-specific oligonucleotide probe to DNA from the individual is indicative of an absence of an alteration in the WDR36 gene that is associated with glaucoma or an increased risk of glaucoma, and is therefore indicative of an absence of WDR36-associated glaucoma or of an WDR36-associated increased risk of glaucoma.

In one embodiment, to detect, screen for, or diagnose the presence of a glaucoma-causing alteration, glaucoma-susceptibility, glaucoma or an increased risk of glaucoma, a hybridization sample is formed by contacting the test sample containing a WDR36 gene with at least one nucleic acid probe. The hybridization sample is maintained under conditions sufficient to allow specific hybridization of the nucleic acid probe to the WDR36 gene. “Specific hybridization”, as used herein, indicates exact hybridization (e.g., with no mismatches). Specific hybridization can be performed under high stringency conditions or moderate stringency conditions, for example.

“Stringency conditions” for hybridization refers to the incubation and wash conditions, e.g., conditions of temperature and buffer concentration, which permit hybridization of a particular nucleic acid to a second nucleic acid; the first nucleic acid may be perfectly (i.e., 100%) complementary to the second, or the first and second may share some degree of complementarity which is less than perfect (e.g., 70%, 75%, 85%, 95%, 98%). For example, certain high stringency conditions distinguish perfectly complementary nucleic acids from those of less complementarity.

“High stringency conditions”, “moderate stringency conditions” and “low stringency conditions” for nucleic acid hybridizations are explained in Current Protocols in Molecular Biology (Ausubel, F. M. et al., “Current Protocols in Molecular Biology”, John Wiley & Sons, (1998)). The exact conditions which determine the stringency of hybridization depend not only on ionic strength (e.g., 0.2×SSC, 0.1×SSC), temperature (e.g., room temperature, 42° C., 68° C.) and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, high, moderate or low stringency conditions can be determined empirically.

By varying hybridization conditions from a level of stringency at which no hybridization occurs to a level at which hybridization is first observed, conditions which will allow a given sequence to hybridize (e.g., selectively) with the most similar sequences in the sample can be determined.

Washing conditions also vary for moderate or low stringency conditions. Washing is the step in which conditions are set so as to determine a minimum level of complementarity of the hybrids. Generally, starting from the lowest temperature at which only homologous hybridization occurs, each ° C. by which the final wash temperature is reduced (holding SSC concentration constant) allows an increase by 1% in the maximum extent of mismatching among the sequences that hybridize. Generally, doubling the concentration of SSC results in an increase in Tₘ of 17° C. Using these guidelines, the washing temperature can be determined empirically for high, moderate or low stringency, depending on the level of mismatch sought.

A low stringency wash comprises washing in a solution containing 0.2×SSC/0.1% SDS for 10 minutes at room temperature; a moderate stringency wash comprises washing in a prewarmed solution (42° C.) solution containing 0.2×SSC/0.1% SDS for 15 minutes at 42° C.; and a high stringency wash comprises washing in prewarmed (68° C.) solution containing 0.1×SSC/0.1% SDS for 15 minutes at 68° C. Furthermore, washes are optionally performed repeatedly or sequentially to obtain a desired result. Equivalent conditions can be determined by varying one or more of the parameters given as an example, as known in the art, while maintaining a similar degree of identity or similarity between the target nucleic acid molecule and the primer or probe used.

In one embodiment, the hybridization conditions for specific hybridization are high stringency. Specific hybridization, if present, is then detected using standard methods. If specific hybridization occurs between the nucleic acid probe and the WDR36 gene in the test sample, then the WDR36 gene has the alteration that is present in the nucleic acid probe. More than one nucleic acid probe can also be used concurrently in this method. Specific hybridization of any one of the nucleic acid probes is indicative of the presence of an alteration in the WDR36 gene that is a glaucoma-causing alteration or glaucoma-susceptibility alteration. The absence of specific hybridization is indicative of the absence of an alteration in the WDR36 gene that is a glaucoma-causing alteration or glaucoma-susceptibility alterations, and is therefore diagnostic for the absence of WDR36-associated glaucoma or the absence of a WDR36-associated increased risk of glaucoma.

Northern analysis can be used to identify the presence or absence of an alteration associated with glaucoma or with an increased risk of glaucoma. For Northern analysis, a test sample of RNA is obtained from the individual by appropriate means. Specific hybridization of a nucleic acid probe, as described above, to RNA from the individual is indicative of the presence of an alteration in the WDR36 gene that is associated with glaucoma or an increased risk of glaucoma, and is therefore diagnostic for WDR36-associated glaucoma or for a WDR36-associated increased risk of glaucoma. Absence of specific hybridization of a nucleic acid probe, as described above, to RNA from the individual is indicative of the absence of an alteration in the WDR36 gene that is associated with glaucoma or an increased risk of glaucoma, and is therefore diagnostic for the absence of WDR36-associated glaucoma or of an WDR36-associate increased risk of glaucoma.

Alternatively, a peptide nucleic acid (PNA) probe is employed instead of a nucleic acid probe in the hybridization methods described above. PNA is a DNA mimic having a peptide-like, inorganic backbone, such as N-(2-aminoethyl)glycine units, with an organic base (A, G, C, T or U) attached to the glycine nitrogen via a methylene carbonyl linker. The PNA probe is designed to specifically hybridize to a gene having an alteration associated with glaucoma. Specific hybridization of a PNA probe, as described above, to RNA from the individual is indicative of the presence of an alteration in the WDR36 gene that is associated with glaucoma or an increased risk of glaucoma, and is therefore diagnostic for WDR36-associated glaucoma or for a WDR36-associated increased risk of glaucoma. Absence of specific hybridization of a PNA probe, as described above, to RNA from the individual is indicative of the absence of an alteration in the WDR36 gene that is associated with glaucoma or an increased risk of glaucoma, and is therefore diagnostic for the absence of WDR36-associated glaucoma or of an WDR36-associate increased risk of glaucoma.

In another method, mutation analysis by restriction digestion can be used to detect mutant genes, or genes containing alterations, if the mutation or alteration in the gene results in the creation or elimination of a restriction site. The D658G mutation in WDR36, for example, creates a new recognition site (Bgl I), thus providing a rapid method for its screening. A test sample containing genomic DNA is obtained from the individual. The polymerase chain reaction (PCR), for example, is used to amplify the WDR36 gene (and, if necessary, the flanking sequences) in the test sample of genomic DNA from the test individual. Restriction fragment length polymorphism (RFLP) analysis is conducted as known in the art. Briefly, amplified DNA is cut into restriction fragments using suitable endonucleases, which only cut the DNA molecule where there are specific DNA sequences, termed recognition sequences, recognized by the enzymes. The restriction fragments are then separated according to length by agarose gel electrophoresis. The distance between the locations cut by restriction enzymes (the restriction sites) varies between individuals: so the length of the fragments varies, and the position of certain gel bands differs between individuals (thus polymorphism). The digestion pattern of the relevant DNA fragment indicates the presence or absence of the mutation or alteration in the WDR36 gene that is associated with glaucoma or an increased risk of glaucoma, and therefore is prognostic or diagnostic for the presence or absence of WDR36-associated glaucoma or WDR36-associated increased risk for glaucoma.

In another embodiment, sequence analysis is employed to detect specific alterations in the WDR36 gene. A test sample of DNA or RNA is obtained from the test individual. PCR or another appropriate methods is used to amplify the gene, and/or its flanking sequences, if desired. The sequence of the WDR36 gene, or a fragment of the gene (e.g., one or more exons), or cDNA, or fragment of the CDNA, or mRNA, or fragment of the mRNA, is determined, using standard methods. The sequence of the gene, gene fragment, cDNA, cDNA fragment, mRNA, or mRNA fragment is compared with the known nucleic acid sequence of the gene, cDNA, or mRNA, as appropriate. The presence of an alteration in the WDR36 gene indicates that the individual has an alteration associated with glaucoma or with an increased risk of glaucoma, and is therefore prognostic or diagnostic for WDR36-associated glaucoma or for a WDR36-associated increased risk of glaucoma. The absence of an alteration in the WDR36 gene indicates that the individual does not have an alteration associated with glaucoma or with an increased risk of glaucoma, and is therefore diagnostic for the absence of WDR36-associated glaucoma or of a WDR36-associated increased risk of glaucoma.

Other methods of nucleic acid analysis can be used to detect alterations in the WDR36 gene. Representative methods include dot blots; direct manual sequencing; automated fluorescent sequencing; single-stranded conformation alteration assays (SSCP); clamped denaturing gel electrophoresis (CDGE); denaturing gradient gel electrophoresis (DGGE), Denaturing high-performance liquid chromatography (DHPLC), High-resolution melting analysis as implemented in LightScanner, mobility shift analysis, restriction enzyme analysis; heteroduplex analysis; chemical mismatch cleavage (CMC); RNAse protection assays; use of polypeptides which recognize nucleotide mismatches, such as E. coli mutS protein; allele-specific PCR, for example.

This disclosure also relates to WDR36 polypeptides, SEQ ID NO: 2. Preferred WDR36 polypeptides are those containing alterations in their amino acid sequences, and wherein the alterations are a glaucoma-causing alteration, or a glaucoma-susceptibility alteration indicative of glaucoma or an increased risk of glaucoma. In addition, also provided are polypeptides corresponding to a portion of SEQ ID NO: 2, referred to as polypeptide fragments. A polypeptide fragment corresponds to about 10 to about 100, specifically 15 to 50, more specifically 20 to 25 contiguous amino acids of SEQ ID NO: 2. In one embodiment, the polypeptide fragment is an immunogenic fragment. As used herein, “immunogenic fragment” refers to a polypeptide fragment that can directly/indirectly induce a specific immune response in appropriate animals or cells and bind with specific antibodies.

An “isolated” or “purified” polypeptide, immunogenic fragment, or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of polypeptide in which the polypeptide is separated from cellular components of the cells from which it is isolated or recombinantly produced. Thus, polypeptide that is substantially free of cellular material includes preparations of polypeptide having less than about 30%, 20%, 10%, or 5% (by dry weight) of heterologous protein (also referred to herein as a “contaminating protein”). When the polypeptide or biologically active portion thereof is recombinantly produced, it is also preferably substantially free of culture medium, i.e., culture medium represents less than about 20%, 10%, or 5% of the volume of the polypeptide preparation. When the polypeptide is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

In another embodiment, determination of the presence or absence of WDR36- associated glaucoma or of a WDR36-associated increased risk of glaucoma is made by examining expression and/or composition of the WDR36 polypeptide. A test sample from an individual is assessed for the presence or absence of an alteration in the expression and/or an alteration in composition of the polypeptide encoded by the WDR36 gene. An alteration in expression of a polypeptide encoded by an WDR36 gene can be, for example, an alteration in the quantitative polypeptide expression (i.e., the amount of polypeptide produced); an alteration in the composition of a polypeptide encoded by an WDR36 gene is an alteration in the qualitative polypeptide expression. Both such alterations can also be present. An “alteration” in the polypeptide expression or composition, as used herein, refers to an alteration in expression or composition in a test sample, as compared with the expression or composition of polypeptide by a WDR36 gene in a control sample. A control sample is a sample that corresponds to the test sample (e.g., is from the same type of cells), and is from an individual who is not affected by glaucoma and who is not at increased risk for glaucoma. An alteration in the expression or composition of the polypeptide in the test sample, as compared with the control sample, is indicative of WDR36-associated glaucoma or of an increased risk of WDR36-associated glaucoma. Absence of an alteration in the expression or composition of the polypeptide in the test sample, as compared with the control sample, is indicative of an absence of WDR36-associated glaucoma or of an increased risk of WDR36-associated glaucoma. Polypeptide alterations include, for example, a P to T change at codon 31 (P31T); an N to S change at codon 355 (N355S); an A to T change at codon 449 (A449T); a C to F change at codon 505 (C505F); an R to Q change at codon 529 (R529Q); a D to N change at codon 548 (D548N); a D to G change at codon 658 (D658G); an L to P change at codon 25 (L25P); an A to V change at codon 163 (A163V); a H to P change at codon 212 (H212P); or a combination comprising one or more of the foregoing alterations.

Suitable means of examining expression or composition of the polypeptide encoded by the WDR36 gene include spectroscopy, colorimetry, electrophoresis, isoelectric focusing, and immunoassays such as immunoblotting. For example, Western blotting analysis, using an antibody that specifically binds to a polypeptide encoded by a mutant WDR36 gene, or an antibody that specifically binds to a polypeptide encoded by a non-mutant gene, can be used to identify the presence in a test sample of a polypeptide encoded by a polymorphic or mutant WDR36 gene, or the absence in a test sample of a polypeptide encoded by a non-polymorphic or non-mutant gene. The presence of a polypeptide encoded by a polymorphic or mutant gene, or the absence of a polypeptide encoded by a non-polymorphic or non-mutant gene, is indicative of an alteration associated with glaucoma or an increased risk of glaucoma, and is therefore prognostic or diagnostic for WDR36-associated glaucoma or for a WDR36-associated increased risk of glaucoma. The absence of a polypeptide encoded by a polymorphic or mutant gene, or the presence of a polypeptide encoded by a non-polymorphic or non-mutant gene, is indicative of the absence of an alteration associated with glaucoma or an increased risk of glaucoma, and is therefore prognostic or diagnostic for an absence of WDR36-associated glaucoma or for an WDR36-associated increased risk of glaucoma.

In one embodiment, the level or amount of polypeptide encoded by an WDR36 gene in a test sample is compared with the level or amount of the polypeptide encoded by the WDR36 gene in a control sample. A level or amount of the polypeptide in the test sample that is higher or lower than the level or amount of the polypeptide in the control sample, such that the difference is statistically significant, is indicative of an alteration in the expression of the polypeptide encoded by the WDR36 gene, and is diagnostic for WDR36-associated glaucoma or for an WDR36-associated increased risk of glaucoma. A level or amount of the polypeptide in the test sample that is not statistically different from the level or amount of the polypeptide in the control sample, is indicative of the absence of an alteration in the expression of the polypeptide encoded by the WDR36 gene, and is diagnostic for an absence of WDR36-associated glaucoma or of an WDR36-associated increased risk of glaucoma.

Alternatively, the composition of the polypeptide encoded by a WDR36 gene in a test sample is compared with the composition of the polypeptide encoded by the WDR36 gene in a control sample. A difference in the composition of the polypeptide in the test sample, as compared with the composition of the polypeptide in the control sample, is diagnostic for WDR36-associated glaucoma or for a WDR36-associated increased risk of glaucoma. An absence of difference in the composition of the polypeptide in the test sample, as compared with the composition of the polypeptide in the control sample, is diagnostic for an absence of WDR36-associated glaucoma or of a WDR36-associated increased risk of glaucoma.

In another embodiment, the level and/or amount and the composition of the polypeptide can be assessed in the test sample and in the control sample. A difference in the amount or level of the polypeptide in the test sample, compared to the control sample; a difference in composition in the test sample, compared to the control sample; or both a difference in the amount or level, and a difference in the composition, is indicative of WDR36-associated glaucoma or an WDR36-associated increased risk of glaucoma. Absence of both a difference in the amount or level, and a difference in the composition, is indicative of an absence of WDR36-associated glaucoma or an WDR36-associated increased risk of glaucoma.

In another embodiment, detection (e.g., screening, prognosis or diagnosis) of the presence or absence of WDR36-associated glaucoma or of a WDR36-associated increased risk of glaucoma is made by examining activity of the WDR36 polypeptide. A test sample from an individual is assessed for the presence or absence of an alteration in the activity of the polypeptide encoded by the WDR36 gene, as compared with the activity of the polypeptide encoded by the WDR36 gene in a control sample. WDR36 may interact with a variety of proteins, including a member of the Cytochrome P450 family (CYP1B1), Optineurin (OPTN) or Myocilin (MYOC). These proteins are referred to herein as “WDR36-interacting polypeptides”.

In one embodiment, an alteration in activity of a polypeptide encoded by a WDR36 gene is, for example, an increase or decrease of interaction between WDR36 polypeptide and an WDR36-interacting polypeptide. The level or amount of WDR36 interaction with a WDR36-interacting polypeptide is assessed in the test sample and in a control sample (for example, a sample comprising native WDR36 polypeptide). A difference in the amount or level of interaction in the test sample, compared to the control sample, is indicative of the presence of an alteration in WDR36, and is thereby indicative of WDR36-associated glaucoma or a WDR36-associated increased risk of glaucoma. Absence of a difference in the amount or level of interaction, is indicative of the absence of such an alteration and thereby indicative of the absence of WDR36-associated glaucoma or a WDR36-associated increased risk of glaucoma.

In another example, the amount or level of activity of a WDR36-interacting polypeptide is used as an indirect measure of the amount or level of activity of WDR36. A difference in the amount or level of activity of the WDR36-interacting polypeptide in the test sample, compared to the control sample, is indicative of the presence of an alteration in WDR36, and is thereby indicative of WDR36-associated glaucoma or a WDR36-associated increased risk of glaucoma. Absence of a difference in the amount or level of activity of the WDR36-interacting polypeptide, is indicative of the absence of such an alteration and thereby indicative of the absence of WDR36-associated glaucoma or a WDR36-associated increased risk of glaucoma. For example, it is possible that WDR36 (either directly or though its interaction with other proteins) can affect the levels of a protein that can be assessed and be used as a proxy for the level of WDR36 activity. An alteration in a test sample of the amount or level of an interacting protein (e.g., an increased amount of the protein), as compared with the amount or level of the protein in a control sample, is indicative of the presence of a mutation in WDR36 and thereby indicative of the presence of WDR36-associated glaucoma or a WDR36-associated increased risk of glaucoma.

Kits useful in the methods of detection, screening, prognosis and/or diagnosis of glaucoma can comprise components useful the methods described herein, including for example, hybridization probes, restriction enzymes (e.g., for RFLP analysis), allele-specific oligonucleotides, antibodies which bind to mutant or to non-mutant (native) WDR36 polypeptide, means for amplification of nucleic acids comprising the WDR36 gene, or means for analyzing the nucleic acid sequence of the WDR36 gene or for analyzing the amino acid sequence of the WDR36 polypeptide, etc. A kit may also comprise a reagent suitable for performing a detection method such as a hybridization reaction, an immunological reaction, and the like.

In another aspect, this disclosure includes an array that includes a substrate having a plurality of addresses. At least one address of the plurality includes a capture probe that binds specifically to a WDR36 nucleic acid or polypeptide comprising an alteration. Suitable capture probes include allele-specific probes. Suitable alterations are those that indicate the presence of WDR36-associated glaucoma or of a WDR36-associated risk of glaucoma in an individual. The array has a density of, for example, about 10, 50, 100, 200, 500, 1,000, 2,000, or 10,000 or more addresses/cm², and ranges between. The plurality of addresses includes 10, 100, 500, 1,000, 5,000, 10,000, 50,000 addresses, for example. Suitable substrates include two-dimensional substrates such as a glass slide, a wafer (e.g., silica or plastic), a mass spectroscopy plate, or a three-dimensional substrate such as a gel pad. Addresses in addition to address of the first plurality are optionally disposed on the array.

In one embodiment, at least one address of the plurality includes a nucleic acid capture probe that hybridizes specifically to a WDR36 polynucleotide, including the sense and/or anti-sense strand. Preferably, the nucleic acid capture probe comprises a WDR36 alteration, such as an allele-specific probe. A subset of addresses of the plurality of addresses includes nucleic acid capture probe for WDR36. Each address of the subset includes a capture probe that hybridizes to a different region of a WDR36 nucleic acid. Addresses of the subset include a capture probe for a WDR36 nucleic acid. Each address of the subset can be unique, overlapping, and/or complementary to a different alteration of WDR36. The array can be used to detect or sequence WDR36 polynucleotides by hybridization.

An array can be generated by various methods, e.g., by photolithographic methods, mechanical methods (e.g., directed-flow methods), pin-based methods, and bead-based techniques.

In another embodiment, at least one address of the plurality includes a polypeptide capture probe that binds specifically to a WDR36 polypeptide or fragment thereof, preferably an WDR36 polypeptide comprising an WDR36 alteration. The polypeptide can be a naturally-occurring interaction partner of the WDR36 polypeptide. In one embodiment, the polypeptide is an antibody, e.g., an antibody such as a monoclonal antibody.

A method of analyzing the expression of WDR36 includes providing an array as described above; contacting the array with a sample and detecting binding of a WDR36 molecule (e.g., nucleic acid or polypeptide) to the array. In one embodiment, the array is a nucleic acid array. Optionally, the method further includes amplifying nucleic acid from the sample prior to or during contact with the array.

In one embodiment, the array is used to assay gene expression in a tissue to ascertain tissue specificity of genes in the array, particularly the expression of WDR36. If a sufficient number of diverse samples are analyzed, clustering (e.g., hierarchical clustering, k-means clustering, Bayesian clustering, and the like) is used to identify other genes that are co-regulated with WDR36. For example, the array can be used for the quantitation of the expression of multiple genes. Thus, not only tissue specificity, but also the level of expression of a battery of genes in the tissue is ascertained. Quantitative data can be used to group (e.g., cluster) genes on the basis of their tissue expression per se and level of expression in that tissue.

In another embodiment, cells are contacted with a therapeutic agent and the expression profile of the cells is determined using the array. The expression profile is compared to the profile of like cells not contacted with the agent. For example, the assay can be used to determine or analyze the molecular basis of an undesirable effect of the therapeutic agent. If an agent is administered therapeutically to treat one cell type but has an undesirable effect on another cell type, the assay can be used to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

The array can be used to monitor expression of one or more genes in the array with respect to time. For example, samples obtained from different time points can be probed with the array. Such analysis can identify and/or characterize the development of a WDR36-associated glaucoma. The method can also evaluate the treatment and/or progression of a WDR36-associated glaucoma.

The array is also useful for ascertaining differential expression patterns of one or more genes in normal and abnormal cells. This approach provides a battery of genes (e.g., including WDR36) that could serve as a molecular target for diagnosis or therapeutic intervention.

The array can be a polypeptide array. At least one address of the plurality has disposed thereon a WDR36 polypeptide or fragment thereof. Methods of producing polypeptide arrays are well-known in the art. Each address of the plurality can have disposed thereon a polypeptide at least 60, 70, 80,85, 90, 95 or 99% identical to a WDR36 polypeptide or fragment thereof. For example, multiple variants of a WDR36 polypeptide (e.g., encoded by allelic variants, site-directed mutants, random mutants, or combinatorial mutants) can be disposed at individual addresses of the plurality. Addresses in addition to the address of the plurality can be disposed on the array.

The polypeptide array can be used to detect a WDR36 binding compound, e.g., an antibody in a sample from a subject with specificity for an WDR36 polypeptide or the presence of an WDR36-binding protein or ligand.

The array is also useful for ascertaining the effect of the expression of a gene on the expression of other genes in the same cell or in different cells (e.g., ascertaining the effect of WDR36 expression on the expression of other genes). This provides, for example, a selection of alternate molecular targets for therapeutic intervention if the ultimate or downstream target cannot be regulated.

In another aspect, a method of analyzing a plurality of probes is disclosed. The method is useful, e.g., for analyzing gene expression. The method includes the use of a two dimensional array having a plurality of addresses, each address of the plurality being positionally distinguishable from each other address of the plurality having a unique capture probe. The array can be contacted with one or more inquiry probes (e.g., probes other than an WDR36 nucleic acid, polypeptide, or antibody), and thereby evaluating the plurality of capture probes. Binding, e.g., in the case of a nucleic acid, hybridization with a capture probe at an address of the plurality, is detected, e.g., by signal generated from a label attached to the nucleic acid, polypeptide, or antibody.

A method of analyzing a plurality of probes in a sample is included. The method is useful, e.g., for analyzing gene expression. The method includes use of a two dimensional array having a plurality of addresses, each address of the plurality being positionally distinguishable from each other address of the plurality having a unique capture probe. The array can be contacted with a first sample from a cell or subject which express or mis-express WDR36 or from a cell or subject in which an WDR36-mediated response has been elicited. The array is then contacted with a second sample in which an WDR36 mediated response has not been elicited, or has been elicited to a lesser extent than in the first sample. Binding of the first sample is compared with the binding of the second sample. Binding, e.g., in the case of a nucleic acid, hybridization with a capture probe at an address of the plurality, can be detected, e.g., by signal generated from a label attached to the nucleic acid, polypeptide, or antibody. The same array can be used for both samples or different arrays can be used. If different arrays are used, the plurality of addresses with capture probes should be present on both arrays.

Genetic mutations in WDR36 can be identified by hybridizing a sample and control nucleic acids, e.g., DNA or RNA, two- dimensional arrays, e.g., chip based arrays. Such arrays include a plurality of addresses, each of which is positionally distinguishable from the other. A different probe may be located at each address of the plurality. A probe can be complementary to a region of a WDR36 nucleic acid or a putative variant (e.g., allelic variant) thereof. A probe can have one or more mismatches to a region of a WDR36 nucleic acid (e.g., a destabilizing mismatch). The arrays can have a high density of addresses, e.g., hundreds or thousands of oligonucleotides probes. For example, genetic mutations in WDR36 can be identified in two-dimensional arrays containing light-generated DNA probes. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In another embodiment, the WDR36 nucleotide or amino acid sequences are provided in a variety of media to facilitate use thereof. Medium refers to a manufacture, other than an isolated nucleic acid or polypeptide molecule, which contains a WDR36 nucleotide or amino acid sequence. Such a manufacture provides the nucleotide or amino acid sequences, or a subset thereof (e.g., a subset of open reading frames (ORFs)) in a form which allows a skilled artisan to examine the manufacture using means not directly applicable to examining the nucleotide or amino acid sequences, or a subset thereof as they exists in nature or in purified form. The manufacture comprises at least one WDR36 nucleic acid or polypeptide sequence that comprises a WDR36 alteration.

A WDR36 nucleotide or amino acid sequence can be recorded on computer readable media. As used herein, “computer readable medium” refers to a medium that can be read and accessed directly by a computer. Such media include, but are not limited to: magnetic storage media, such as floppy discs, hard disc storage media, and magnetic tape; optical storage media such as CD-ROM; electrical storage media such as RAM and ROM; and hybrids of these categories such as magnetic/optical storage media. The skilled artisan will readily appreciate how the presently known computer readable media can be used to create a manufacture comprising a computer readable medium having recorded thereon a nucleotide or amino acid sequence of the present invention.

As used herein, “recorded” refers to a process for storing information on computer readable media. The skilled artisan can readily adopt the presently known methods for recording information on computer readable media to generate manufactures comprising WDR36 nucleotide or amino acid sequence information.

A variety of data storage structures are available to a skilled artisan for creating a computer readable medium having recorded thereon a WDR36 nucleotide or amino acid sequence. The choice of the data storage structure will generally be based on the means chosen to access the stored information. In addition, a variety of data processor programs and formats can be used to store the nucleotide sequence information on computer readable medium. The sequence information can be represented in a word processing text file, formatted in commercially-available software such as WordPerfect and MicroSoft Word, or represented in the form of an ASCII file, stored in a database application, such as DB2, Sybase, Oracle, or the like. The skilled artisan can readily adapt a number of data processor structuring formats (e.g., text file or database) in order to obtain a computer readable medium having recorded thereon the nucleotide sequence information.

By providing WDR36 nucleotide or amino acid sequences in computer readable form, the skilled artisan can routinely access the sequence information for a variety of purposes. For example, one skilled in the art can use the WDR36 nucleotide or amino acid sequences in computer readable form to compare a target sequence or target structural motif with the sequence information stored within the data storage means. Search means are used to identify fragments or regions of the WDR36 sequences that match a particular target sequence or target motif.

Suitable target sequences include nucleotide or amino acid sequences of six or more nucleotides or two or more amino acids. A skilled artisan can readily recognize that the longer a target sequence is, the less likely a target sequence will be present as a random occurrence in the database. Suitable sequence lengths of a target sequence are about 10 to about 1000 amino acids, or about 30 to about 300 nucleotide residues. However, it is well recognized that commercially important fragments, such as sequence fragments involved in gene expression and protein processing, may be of shorter length.

A target structural motif or target motif refers to a rationally selected sequence or combination of sequences in which the sequence(s) are chosen based on a three-dimensional configuration which is formed upon the folding of the target motif. There are a variety of target motifs known in the art. Protein target motifs include, but are not limited to, enzyme active sites and signal sequences. Nucleic acid target motifs include, but are not limited to, promoter sequences, hairpin structures and inducible expression elements (protein binding sequences).

Computer software is publicly available which allows a skilled artisan to access sequence information provided in a computer readable medium for analysis and comparison to other sequences. A variety of known algorithms are disclosed publicly and a variety of commercially available software for conducting search means are and can be used in computer-based systems. Examples of such software include, but are not limited to, MacPattern (EMBL), BLASTN and BLASTX (NCBIA).

For example, software which implements the BLAST and BLAZE search algorithms on a Sybase system can be used to identify open reading frames (ORFs) of the sequences of the invention which contain homology to ORFs or proteins from other libraries. Such ORFs are protein encoding fragments and are useful in producing commercially important proteins such as enzymes used in various reactions and in the production of commercially useful metabolites.

The present invention also includes methods of treatment (prophylactic and/or therapeutic) for glaucoma or for an increased risk of glaucoma, using a WDR36 therapeutic agent. The methods can be used for individuals diagnosed with, or suspected of having, WDR36-associated glaucoma or a WDR36-associated increased risk of glaucoma. The methods can also be used for individuals diagnosed with or suspected of having glaucoma or an increased risk of glaucoma other than those associated with WDR36, as they may similarly be beneficial in such individuals by altering the course of the glaucoma. WDR36-interacting polypeptides are appropriate targets for WDR36 therapeutic agents, to alter the activity and interaction between them and WDR36 and thereby treat glaucoma.

A “WDR36 therapeutic agent” is an agent used for the treatment of glaucoma, that alters (e.g., enhances or inhibits) WDR36 polypeptide activity and/or WDR36 gene expression (e.g., an WDR36 agonist or antagonist). The therapy is designed to inhibit, alter, replace or supplement activity of the mutant WDR36 polypeptide in an individual, or to inhibit, alter, replace or supplement activity of a WDR36-interacting polypeptide in an individual.

An WDR36 therapeutic agent can alter WDR36 activity or gene expression by a variety of means, such as, for example, by providing additional protein or by upregulating the transcription or translation of WDR36; by altering posttranslational processing of the WDR36 polypeptide; by altering transcription of splicing variants of WDR36; or by altering WDR36 polypeptide activity (e.g., by binding to WDR36), or by altering (upregulating or downregulating) the transcription or translation of WDR36. Other WDR36 therapeutic agents can target WDR36-interacting polypeptides, to alter activity or expression of genes encoding WDR36-interacting polypeptides or of other genes in the pathways in which WDR36 takes part.

Representative WDR36 therapeutic agents include several different classes of agents such as, for example, nucleic acids, proteins, polypeptides, antibodies, small molecules, and combinations comprising one or more of the foregoing molecules.

In one embodiment, the WDR36 therapeutic agent is a nucleic acid, such as a gene, cDNA, mRNA, a nucleic acid encoding an WDR36 polypeptide (e.g., SEQ ID NO: 1) or a variant of WDR36, wherein a nucleic acid encoding a variant (a variant nucleic acid molecule) is one that is not necessarily found in nature but which encodes a polypeptide having the amino acid sequence of WDR36. Thus, for example, DNA molecules that comprise a sequence that is different from the naturally-occurring nucleotide sequence but which, due to the degeneracy of the genetic code, encode WDR36, are contemplated, as are nucleotide sequences encoding portions (fragments), or encoding variant polypeptides such as analogues or derivatives of WDR36. Such variants can be naturally-occurring, such as in the case of allelic variation or single nucleotide polymorphisms, or non-naturally-occurring, such as those induced by various mutagens and mutagenic processes. Intended variations include, but are not limited to, addition, deletion and substitution of one or more nucleotides that can result in conservative or non-conservative amino acid changes, including additions and deletions. Preferably the nucleotide (and/or resultant amino acid) changes are silent or conserved; that is, they do not alter the characteristics or activity of the mutant WDR36 (e.g., the ability to interact with other specific proteins, as described in detail below). Other alterations of the nucleic acid molecules can include, for example, labelling, methylation, internucleotide modifications such as uncharged linkages (e.g., methyl phosphonates, phosphotriesters, phosphoamidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids). Also included are synthetic molecules that mimic nucleic acid molecules in the ability to bind to designated sequences via hydrogen bonding and other chemical interactions. Such molecules include, for example, those in which peptide linkages substitute for phosphate linkages in the backbone of the molecule.

Other WDR36 therapeutic agents include aptamers, which are DNA or RNA molecules that have been selected based on their ability to bind other molecules, e.g., aptamers.

In another embodiment, the WDR36 therapeutic agent is a WDR36 polypeptide (e.g., SEQ ID NO: 2), a peptidomimetic, a portion of an WDR36 polypeptide, or a derivative of an WDR36 polypeptide, or another splicing variant encoded by the WDR36 gene or fragments or derivatives thereof. Fusion proteins or other polypeptides comprising fragments (particularly fragments retaining an activity of WDR36) can be employed, as can WDR36 polypeptides encompassing sequencing variants.

Active fragments perform one or more of the same functions as the whole WDR36 polypeptide. For example, active fragments comprise a domain, segment, or motif that has been identified by analysis of the protein sequence using well-known methods. Active fragments can be discrete (not fused to other amino acids or polypeptides) or can be within a larger polypeptide. Further, several fragments can be comprised within a single larger polypeptide.

Variants include a substantially homologous polypeptide encoded by the same genetic locus in an organism, i.e., an allelic variant, as well as other splicing variants. Variants also encompass polypeptides derived from other genetic loci in an organism, but having significant homology to a polypeptide encoded by a WDR36 gene or nucleic acid as described above. Variants also include proteins substantially homologous or identical to these proteins but derived from another organism, i.e., an ortholog. Variants also include proteins that are substantially homologous or identical to these proteins that are produced by chemical synthesis. Variants also include proteins that are substantially homologous or identical to these proteins and that are produced by recombinant methods. Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gin, exchange of the basic residues Lys and Arg and replacements among the aromatic residues Phe and Tyr. A variant polypeptide can differ in amino acid sequence by one or more substitutions, deletions, insertions, inversions, fusions, and truncations or a combination of any of these. Further, variant polypeptides can be fully functional or can lack function in one or more activities. Fully functional variants typically contain only conservative variation or variation in non-critical residues or in non-critical regions. Functional variants can also contain substitution of similar amino acids that result in no change or an insignificant change in function. Alternatively, such substitutions may positively or negatively affect function to some degree Non-functional variants typically contain one or more non-conservative amino acid substitutions, deletions, insertions, inversions, or truncation or a substitution, insertion, inversion, or deletion in a critical residue or critical region. Amino acids that are essential for function can be identified by methods known in the art, such as site-directed mutagenesis or alanine-scanning mutagenesis. Sites that are critical for polypeptide activity can also be determined by structural analysis such as crystallization, nuclear magnetic resonance or photoaffinity labelling.

In another embodiment, the WDR36 therapeutic agent is an antibody (e.g., an antibody to a mutant WDR36 polypeptide, an antibody to a non-mutant WDR36 polypeptide, or an antibody to a particular splicing variant of the WDR36 polypeptide); a ribozyme; a peptidomimetic; a small molecule or other agent that alters WDR36 polypeptide activity and/or gene expression (e.g., which upregulate or downregulate expression of the WDR36 gene); or another agent that alters (e.g., enhance or inhibit) WDR36 gene expression or WDR36 polypeptide activity, that alters posttranslational processing of the WDR36 polypeptide, or that regulates transcription of WDR36 splicing variants (e.g., agents that affect which splicing variants are expressed, or that affects the amount of each splicing variant that is expressed).

For example, an antibody to a mutant WDR36 polypeptide can be used to inhibit an activity of the mutant protein. The term “antibody”, as used herein, refers to immunoglobulin molecules and immunologically active portions of immunoglobulin molecules, i.e., molecules that contain an antigen binding site that specifically binds an antigen. A molecule that specifically binds to a polypeptide is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. Either polyclonal or monoclonal antibodies can be used. The term “monoclonal antibody” or “monoclonal antibody composition”, as used herein, refers to a population of antibody molecules that contain only one species of an antigen binding site capable of immunoreacting with a particular epitope of a protein (e.g., the mutant WDR36). A monoclonal antibody composition thus typically displays a single binding affinity for a particular polypeptide with which it immunoreacts.

Polyclonal antibodies can be prepared as described above by immunizing a suitable subject with a desired immunogen, e.g., WDR36 polypeptide or fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique, the EBV-hybridoma technique, or trioma techniques. The technology for producing hybridomas is well known. Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds the protein of interest.

Alternative to preparing monoclonal antibody-secreting hybridomas, a monoclonal antibody can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available. Additionally, examples of methods and reagents particularly amenable for use in generating and screening antibody display library are readily available in the art.

Additionally, recombinant antibodies, such as chimeric and humanized monoclonal antibodies, comprising both human and non-human portions, which can be made using standard recombinant DNA techniques, can be used. Such chimeric and humanized monoclonal antibodies can be produced by recombinant DNA techniques known in the art.

More than one WDR36 therapeutic agent can be used concurrently, if desired. The WDR36 therapeutic agent(s) are administered in a therapeutically effective amount (i.e., an amount that is sufficient to treat the disease, such as by ameliorating symptoms associated with the disease, preventing or delaying the onset of the disease (e.g., particularly for an individual at increased risk for glaucoma), and/or also lessening the severity or frequency of symptoms of the disease). The amount which will be therapeutically effective in the treatment of a particular disorder or condition will depend on the nature of the disorder or condition, and can be determined by standard clinical techniques. In addition or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

The term, “treatment” refers not only to ameliorating symptoms associated with the disease, but also preventing or delaying the onset of the disease and lessening the severity or frequency of symptoms of the disease. Thus, “treatment of glaucoma” refers not only to treatment after appearance of symptoms of glaucoma (therapeutic treatment), but also to prophylactic treatment (prior to appearance of symptoms). Treatment may be particularly beneficial for individuals in whom an increased risk of glaucoma has been identified, as it may delay onset of the disease, or prevent symptoms of the disease entirely. Thus, treatment can be used not only for individuals having glaucoma, but those at risk for developing glaucoma (e.g., those at increased risk for glaucoma, such as those having an alteration in the WDR36 gene that is associated with increased risk of glaucoma).

In one embodiment, a nucleic acid is used in the treatment of glaucoma. The nucleic acid as described above can be used, either alone or in a pharmaceutical composition as described above. For example, the WDR36 gene or a cDNA encoding the WDR36 polypeptide, either by itself or included within a vector, can be introduced into cells (either in vitro or in vivo) such that the cells produce native WDR36 polypeptide. In another example, a gene encoding a WDR3 6-interacting polypeptide or a cDNA encoding the WDR36-interacting polypeptide, either by itself or included within a vector, can be introduced into cells (either in vitro or in vivo) such that the cells produce native WDR36-interacting polypeptide. If necessary, cells that have been transformed with the gene or cDNA or a vector comprising the gene or cDNA can be introduced (or re-introduced) into an individual affected with the disease. Thus, cells that in nature lack native expression and activity of the polypeptide, or have mutant expression and activity, can be engineered to express the desired polypeptide (e.g., WDR36 polypeptide, or, for example, an active fragment of the WDR36 polypeptide). In one embodiment, a nucleic acid encoding the WDR36 polypeptide, or an active fragment or derivative thereof, can be introduced into an expression vector, such as a viral vector, and the vector can be introduced into appropriate cells which lack native WDR36 expression in an animal. For example, for the treatment of glaucoma, the vector comprising the nucleic acid can be introduced intraocularly. In such methods, a cell population can be engineered to inducibly or constitutively express active WDR36 polypeptide. Other gene transfer systems, including viral and nonviral transfer systems, can be used. Alternatively, nonviral gene transfer methods, such as calcium phosphate coprecipitation, mechanical techniques (e.g., microinjection); membrane fusion-mediated transfer via liposomes; or direct DNA uptake, can also be used.

Alternatively, in another embodiment, a WDR36 nucleic acid, or a nucleic acid complementary to such a nucleic acid, can be used in “antisense” therapy, in which a nucleic acid (e.g., an oligonucleotide) which specifically hybridizes to the mRNA and/or genomic DNA of the WDR36 gene (or to the mRNA and/or genomic DNA of a gene encoding an WDR36-interacting polypeptide) is administered or generated in situ. The antisense nucleic acid that specifically hybridizes to the mRNA and/or DNA can inhibit the expression of the WDR36 polypeptide or of the WDR36-interacting polypeptide, e.g., by inhibiting translation and/or transcription. Binding of the antisense nucleic acid can be by conventional base pair complementarity, or, for example, in the case of binding to DNA duplexes, through specific interaction in the major groove of the double helix.

An antisense construct can be delivered, for example, as an expression plasmid as described above. When the plasmid is transcribed in the cell, it produces RNA that is complementary to a portion of the mRNA and/or DNA that encodes WDR36 polypeptide (or which encodes WDR36-interacting polypeptide). Alternatively, the antisense construct is an oligonucleotide probe which is generated ex vivo and introduced into cells; it then inhibits expression by hybridizing with the mRNA and/or genomic DNA. In one embodiment, the oligonucleotide probes are modified oligonucleotides that are resistant to endogenous nucleases, e.g., exonucleases and/or endonucleases, thereby rendering them stable in vivo. Exemplary nucleic acid molecules for use as antisense oligonucleotides are phosphoramidate, phosphothioate and methylphosphonate analogs of DNA. With respect to antisense DNA, oligodeoxyribonucleotides derived from the translation initiation site, e.g., between the −10 and +10 regions of the WDR36 gene sequence, are preferred.

To perform antisense therapy, oligonucleotides (mRNA, CDNA or DNA) are designed that are complementary to mRNA encoding WDR36 (or encoding a WDR36-interacting polypeptide). The antisense oligonucleotides bind to mRNA transcripts and prevent translation. Absolute complementarity, although preferred, is not required. A sequence “complementary” to a portion of an RNA, as referred to herein, indicates that a sequence has sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid, as described in detail above. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures.

In one embodiment, oligonucleotides complementary to either the 5′ or 3′ untranslated, non-coding regions of the WDR36 gene (or the gene encoding the WDR36-interacting polypeptide) can also be used in an antisense approach to inhibit translation of endogenous mRNA. Oligonucleotides complementary to the 5′ untranslated region of the mRNA can include the complement of the AUG start codon. Antisense oligonucleotides complementary to mRNA coding regions can also be employed. While antisense oligonucleotides complementary to the can region sequence can be used, those complementary to the transcribed untranslated region can also be used. Whether designed to hybridize to the 5′, 3′ or coding region of WDR36 mRNA, antisense nucleic acids are suitably at least six nucleotides in length, and are more specifically oligonucleotides ranging from 6 to about 50 nucleotides in length. In certain embodiments, the oligonucleotide is at least 10 nucleotides, at least 18 nucleotides, at least 24 nucleotides, or at least 50 nucleotides.

If desired, in vitro studies can be performed to quantitate the ability of the antisense oligonucleotide to inhibit gene expression. These studies utilize controls that distinguish between antisense gene inhibition and nonspecific biological effects of oligonucleotides. These studies can compare levels of the target RNA or protein with that of an internal control RNA or protein. In a preferred embodiment, the control oligonucleotide is of approximately the same length as the test oligonucleotide and the nucleotide sequence of the oligonucleotide differs from the antisense sequence so much so as to prevent specific hybridization to the target sequence.

The oligonucleotides used in antisense therapy can be DNA, RNA, or chimeric mixtures or derivatives or modified versions thereof, single-stranded or double-stranded. The oligonucleotides can be modified at the base moiety, sugar moiety, or phosphate backbone, for example, to improve stability of the molecule, hybridization, etc. The oligonucleotides can include other appended groups such as peptides (e.g., for targeting host cell receptors in vivo), or agents facilitating transport across the cell membrane or the blood-brain barrier, or hybridization- triggered cleavage agents or intercalating agents. To this end, the oligonucleotide may be conjugated to another molecule, e.g., a peptide, hybridization triggered cross-linking agent, transport agent, hybridization-triggered cleavage agent, etc.

Ribozyme molecules designed to catalytically cleave WDR36 mRNA transcripts can also be used to prevent translation of WDR36 mRNA and expression of WDR36 polypeptide, particularly, for example, to prevent translation of a mutant WDR36 polypeptide. Alternatively, they can be designed to catalytically cleave mRNA transcripts of genes encoding WDR36-interacting polypeptides. Ribozymes are enzymatic RNA molecules capable of catalyzing the specific cleavage of RNA. The mechanism of ribozyme action involves sequence specific hybridization of the ribozyme molecule to complementary target RNA, followed by an endonucleolytic cleavage. The composition of ribozyme molecules includes one or more sequences complementary to the target gene mRNA, and includes the catalytic sequence responsible for mRNA cleavage. Ribozymes that cleave mRNA at site specific recognition sequences can be used to destroy WDR36 mRNAs. As in the antisense approach, the ribozymes can be composed of modified oligonucleotides (e.g. for improved stability, targeting, etc.) and can be delivered to cells which express WDR36 in vivo (e.g., ocular cells).

Endogenous WDR36 gene expression, particularly mutant WDR36 gene expression, can also be reduced by inactivating or “knocking out” the WDR36 gene or its promoter, or the gene or promoter of an WDR36-interacting polypeptide, using targeted homologous recombination. For example, a non-functional WDR36 gene (or a completely unrelated DNA sequence) flanked by DNA homologous to the endogenous WDR36 gene (either the coding regions or regulatory regions of the WDR36 gene) can be used, with or without a selectable marker and/or a negative selectable marker, to transfect cells that express WDR36 in vivo. Insertion of the DNA construct, via targeted homologous recombination, results in inactivation of the WDR36 gene. Similar methods can be used for genes encoding WDR36-interacting polypeptides. The recombinant DNA constructs can be directly administered or targeted to the required site in vivo using appropriate vectors, as described above. Alternatively, expression of non-mutant WDR36 or of WDR36-interacting polypeptides can be increased using a similar method: targeted homologous recombination can be used to insert a DNA construct comprising a non-mutant, functional gene in place of a mutant gene in the cell, as described above.

Alternatively, endogenous WDR36 gene expression, or expression of a gene encoding an WDR36-interacting polypeptide, can be reduced by targeting deoxyribonucleotide sequences complementary to the regulatory region of the gene (i.e., the promoter and/or enhancers) to form triple helical structures that prevent transcription of the WDR36 gene in target cells in the body. Likewise, the antisense constructs, by antagonizing the normal biological activity of one of the WDR36 polypeptides, can be used in the manipulation of tissue, e.g. tissue differentiation, both in vivo and for ex vivo tissue cultures. Furthermore, the anti-sense techniques (e.g. microinjection of antisense molecules, or transfection with plasmids whose transcripts are anti-sense with regard to a WDR36 mRNA or gene sequence) can be used to investigate role of WDR36 in developmental events, as well as the normal cellular function of WDR36 in adult tissue. Such techniques can be utilized in cell culture, but can also be used in the creation of transgenic animals.

In yet another embodiment, polypeptides and/or agents that alter (e.g., enhance or inhibit) WDR36 polypeptide activity, as described herein, can be used in the treatment or prevention of glaucoma. Polypeptides and/or agents that alter (e.g., enhance or inhibit) activity of WDR36-interacting polypeptides, as described herein, can also be used in the treatment or prevention of glaucoma. The polypeptides or agents can be delivered in a composition, as described above, or by themselves. They can be administered systemically, or can be targeted to a particular tissue (e.g., eye tissue). The proteins and/or agents can be produced by a variety of means, including chemical synthesis; recombinant production; in vivo production.

A combination of any of the above methods of treatment (e.g., administration of non-mutant WDR36 polypeptide in conjunction with antisense therapy targeting mutant WDR36 mRNA), can also be used.

WDR36 therapeutic agents are administered to individuals to treat (prophylactically or therapeutically) glaucoma. In conjunction with such treatment, the pharmacogenomics (i.e., the study of the relationship between an individual's genotype and that individuals response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of a polypeptide, expression of a nucleic acid, or mutation content of the WDR36 gene in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. In general, two types of pharmacogenetic conditions can be differentiated. Genetic conditions transmitted as a single factor altering the way drugs act on the body are referred to as “altered drug action”. Genetic conditions transmitted as single factors altering the way the body acts on drugs are referred to as “altered drug metabolism”. These pharmacogenetic conditions can occur either as rare defects or as polymorphisms.

Thus, the activity of a WDR36 polypeptide, expression of an WDR36 nucleic acid encoding the polypeptide, or mutation content of an WDR36 gene in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of activity or expression of the polypeptide, such as an WDR36 therapeutic agent.

The methods of treatment described above utilize agents which can be incorporated into pharmaceutical compositions, if desired. For instance, a protein or protein, fragment, fusion protein or prodrug thereof, or a nucleotide or nucleic acid construct (vector) comprising a nucleic acid encoding WDR36, or an agent that alters WDR36 activity, can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitoi, lactose, starch, magnesium stearate, polyvinyl pyrollidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. In a preferred embodiment, the composition is introduced intraocularly (e.g., eye drops). Other suitable methods of introduction can also include gene therapy (as described below), rechargeable or biodegradable devices, particle acceleration devises (“gene guns”) and slow release polymeric devices. The pharmaceutical compositions can also be administered as part of a combinatorial therapy with other agents.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The agents are administered in a therapeutically effective amount. The amount of agents which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

The invention also provides a pharmaceutical pack or kit comprising one or more containers filled with one or more of the ingredients of the pharmaceutical compositions that can be used in the methods of treatment. Optionally associated with such container(s) can be a notice in the form prescribed by a governmental agency regulating the manufacture, use or sale of pharmaceuticals or biological products, which notice reflects approval by the agency of manufacture, use of sale for human administration. The pack or kit can be labelled with information regarding mode of administration, sequence of drug administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the agents can be separated, mixed together in any combination, present in a single vial or tablet. Agents assembled in a blister pack or other dispensing means is preferred. For the purpose of this invention, unit dosage is intended to mean a dosage that is dependent on the individual pharmacodynamics of each agent and administered in FDA approved dosages in standard time courses.

In another embodiment, included is the production of nonhuman transgenic animals. For example, in one embodiment, a host cell comprising a nucleic acid encoding WDR36 (e.g., a fertilized oocyte or an embryonic stem cell into which a nucleic acid molecule encoding WDR36 polypeptide) is used. Such host cells can be used to create nonhuman transgenic animals in which exogenous nucleotide sequences have been introduced into the genome or homologous recombinant animals in which endogenous nucleotide sequences have been altered. Alternatively, the invention also pertains to production of nonhuman animals in which the native WDR36 has been altered.

Such animals are useful for studying the function and/or activity of the nucleotide sequence and polypeptide encoded by the sequence and for identifying and/or evaluating modulators of their activity, in order to investigate the processes of WDR36-associated glaucoma. As used herein, a “transgenic animal” is a nonhuman animal, preferably a mammal, more preferably a rodent such as a rat or mouse, or a primate, in which one or more of the cells of the animal includes a transgene. Other examples of transgenic animals include nonhuman primates, sheep, dogs, cows, goats, chickens and amphibians. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a nonhuman animal, preferably a mammal, more preferably a mouse, in which an endogenous gene has been altered by homologous recombination between the endogenous gene and an exogenous DNA molecule (e.g., a mutated form of the endogenous gene) introduced into a cell of the animal, e.g., an embryonic cell of the animal, prior to development of the animal.

Methods for generating transgenic animals via embryo manipulation and microinjection, particularly animals such as mice, have become conventional in the art.

The identification of WDR36 gene's association with glaucoma, and its known interaction with a group of proteins, provides the first opportunity to examine biochemical pathways involved in the etiology of this group of eye disorders. Furthermore, identification of the gene as a significant contributing factor to the development of glaucoma allows screening for this disorder in high risk individuals, such as the elderly population, as well as prophylactic and therapeutic treatment of the disease.

The following Examples are offered for the purpose of illustrating the present invention and is not to be construed to limit the scope of this invention.

### EXAMPLES

### Example 1

### Positional Mapping of a New Locus (GLC1G) for Adult-Onset POAG

Family Ascertainment:

The clinical status of all affected POAG members of studied families have previously been confirmed by their respective caring ophthalmologists. The diagnoses of glaucoma agreed with the standard criteria and included abnormal cup/disc ratio and glaucomatous visual field loss with or without increased IOP. The original GLC1G-linked family of POAG-527 has a total of 108 members in five generations of whom 54 (7 affected, 5 glaucoma suspect and 42 unaffected) participated in this study. The other large family of POAG-002 that was originally used in the genome scan, but subsequently proven to be unlinked to the GLC1G locus has a total of 99 members in four generations, 40 of whom have been sampled previously (13 affected, 4 ocular hypertension and 23 unaffected). The clinical status of all other unrelated familial and sporadic POAG subjects screened during this study have previously been confirmed.

Genome-Wide Scan:

A genome-wide scan was carried out with two different sets of fluorescently end-labeled primers (ABI and CHLC-Weber linkage mapping sets). The amplified PCR products were loaded onto a 96-well gel and run on an ABI-377 automated DNA sequencer. Computer assisted data collection and genotyping were performed with the ABI GENESCANC® analysis and ABI GENOTYPER® computer software. The obtained genotypic data were analyzed and further used for additional linkage analysis and saturation mapping.

Saturation Mapping and Refinement of the GLC1G Critical Region:

All genotypes either from the ABI or CHLC-Weber florescent linkage mapping sets were automatically transferred into an in-house database management system (DMS) program. Pedigrees and their genotypic data for each of the 22-chromosomes were subsequently exported to the CYRILLIC program and haplotypes were constructed for each pedigree and for each chromosome. Co-inheritace of affected-bearing haplotypes with the POAG phenotype was inspected manually for each of the studied families. Using additional Short Tandem Repeat Polymorphism (STRP) markers potential regions of interest were saturated in these families.

After a hint of linkage was observed on chromosome 5q, fluorescent genotyping and saturation mapping of other families with new STRP markers were carried out in order to reduce the identified interval even further. The 2-point and multi-point LOD scores were calculated with the MLINK and LINKMAP components of the LINKAGE program, respectively.

Initially, genome scans of two large POAG families (i.e., POAG-002 and POAG-527) with 20 affected and 9 glaucoma suspects were used to obtain a provisional hint of linkage to the 5q33-q35 region. Extended saturation mapping established critical recombination in 2 of the affected individuals in POAG-002 and, therefore, excluded genetic linkage of this family to this region of 5q. However, additional genotyping in POAG-527 revealed consistent sharing of an extended affected haplotype from 5q21.3 (D5S1466) to 5q35.2 (D5S498) in all 7 affected members of this family. Therefore, in light of this new linkage information and due to a discrepancy that currently exists between genetic linkage and physical map position of several DNA markers from this region of chromosome 5 (including D5S1466), the upper physical boundary for this new POAG locus is now 5q21.3 and not 5q33 as initially envisaged.

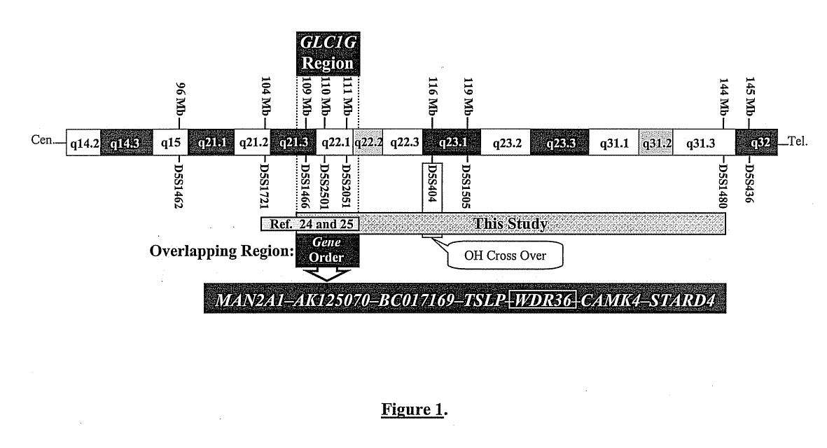

After a hint of linkage was observed with the 5q region, seven additional POAG families with a total of 194 individuals and 31 living affected subjects were genotyped. Only two of these families were consistent with linkage to the same 5q region. Further saturation mapping in these families confined this new linkage to an interval of approximately 35-Mb (FIG. 1) that is flanked by D5S1466 (Sq2l.3) and D5S1480 (5q31.3). Two flanking markers of D5S1466 (109-Mb from the top of chromosome 5) and D5S 1480 (144-Mb) and a further cross over with D5S404 (116-Mb) in a subject with Ocular Hypertension (OH) were discovered. Another study identified a region between D5S1721 (104-Mb) and D5S2051 (111-Mb). An overlapping region of approximately 2 Mb between D5S 1466 and D5S2051 defined the GLC1G locus. Additional recombination in a subject with ocular hypertension (OH) and for DNA marker of D5S404 (5q23.1) suggested that the identified genetic linkage may be limited to approximately 7-Mb (FIG. 1). In a parallel study, a group of 638 individuals (including >400 affected subjects) in 139 POAG families were being screened for the entire genome. As this screening also covered this newly identified region on 5q, a number of new families showed potential linkage to this region. Altogether, a total of 7 families were consistent with linkage to this region of 5q. Table 1 shows individual and multipoint LOD score values for three closely linked DNA markers of D5S1462, D5S2501 and D5S1505 (FIG. 1). This newly identified POAG locus has now been designated as GLC1G by HUGO Gene Nomenclature Committee (HGNC). Recently, other groups have reported linkage in a single adult-onset POAG family and, within a 6.6-Mb region (FIG. 1) that is flanked by D5S1721 (5q21.2) and D5S2051 (5q22.1). Taken together, these two overlapping linkage data defined the critical region of the GLC1G locus between D5S1466 (5q21.3) and D5S2051 (5q22.1) and within a region of approximately 2-Mb (FIG. 1). This newly identified GLC1G locus provided an opportunity for rapid mutation screening and identification of the defective gene at this locus.

TABLE 1
Two-point and Multipoint LOD Score Values for 3 Closely GLC1G-Linked Markers

| DNA | Recombination Fractions (cM) | | | | | | | | | | | |
|---|---|---|---|---|---|---|---|---|---|---|---|---|
| Markers | 0.00 | 0.05 | 0.10 | 0.15 | 0.20 | 0.25 | 0.30 | 0.35 | 0.40 | 0.45 | Zₘₐₓ | θₘₐₓ |
| | −∞ | 2.25 | 2.22 | 1.99 | 1.68 | 1.33 | 0.96 | 0.60 | 0.29 | 0.08 | 2.27 | 0.07 |
| D5S2501 | 5.41 | 4.93 | 4.39 | 3.81 | 3.19 | 2.54 | 1.87 | 1.20 | 0.60 | 0.15 | 5.41 | 0.00 |
| D5S1505 | 2.45 | 2.32 | 2.10 | 1.82 | 1.50 | 1.16 | 0.82 | 0.50 | 0.24 | 0.06 | 2.45 | 0.00 |
| Multi-point | 4.90 | 4.70 | 4.44 | 4.05 | 3.61 | 3.13 | 2.61 | 2.07 | 1.51 | 0.96 | 4.90 | 0.00 |

### Example 2

### GLC1G Candidate Genes Screening

Mutation Screening:

After linkage to the GLC1G locus was established and its candidate region was reduced to a region of approximately 2-Mb, all the 7 known genes mapping to the critical region (FIG. 1) were selected for mutation screening. Primers were designed to flank intron-exon boundaries of the selected genes and PCR amplification performed using genomic DNA of affected individuals (primer sequences are available on request). Direct sequencing carried out with ABI-Big Dye Terminator Cycle sequencing kit and run on an ABI-3100 Genetic Analyzer and DNA Sequencer.

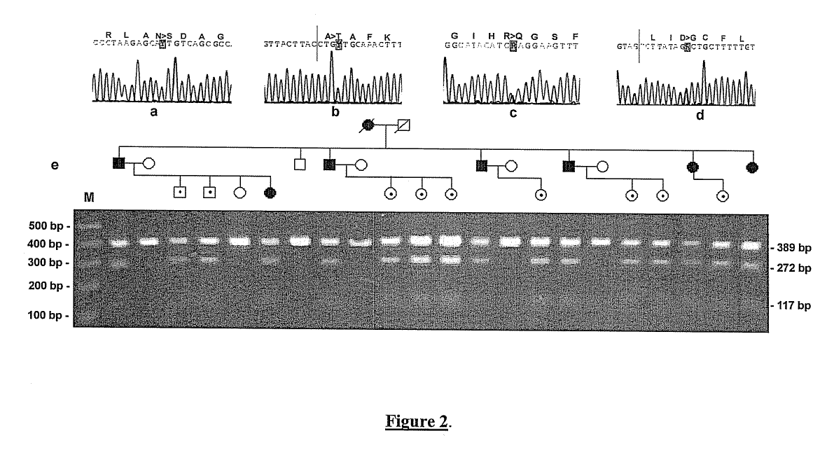

The critical region of the GLC1G locus contains 7 known genes (MAN2A1, AK]25070, BC07169, TSLP, WDR36, CAMK4 and STARD4) and at least 3 other predicted genes (LOC153778, LOC441101, LOC441102). For each of the 7 known genes (FIG. 1), a series of genomic primers were made and their respective coding exons were PCR amplified and screened for mutation by direct DNA sequencing. Analysis of genomic DNA from at least 2 affected subjects of the original GLC1G-linked family of POAG-527 revealed 4 sequence alterations in only 2 genes. Three of these variations (i.e., IVS6−5A>G, S337S and A413V in CAMK4) were polymorphisms and only one sequence change in WDR36 proven to be significant. The observed variation in this gene was a single heterozygous DNA alteration in exon 17 (c. 1973A>G; GAC>GGC) that is predicted to change Aspartic Acid (acidic) to Glycine (neutral and polar) at amino acid 658 (D658G). This mutation (FIG. 2d) was observed in 7 affected (with mean age of 63.7 years) and 9 asymptomatic gene carriers (with mean age of 40.4 years) but it was absent in 9 normal members of this family (with mean age of 45.3 years) and another 6 married-in normal spouses (FIG. 2e). Screening of a total of 476 normal control chromosomes by restriction enzyme digestion (Bgl I) showed that this mutation is absent in all the normal subjects tested. Direct sequencing of WDR36 in another 129 unrelated affected glaucoma probands (including the 6 GLC1G-linked families) identified additional 23 DNA alterations (Table 2). Three of these alterations (N355S, A449T, and R529Q) (FIG. 2a-c) were considered disease causing, as they were not present in any of the public databases and, in a group of at least 200 tested chromosomes from normal control subjects. The genomic DNA of at least one affected subject from each of the original 7 linked families was directly sequenced. However, 2 of these families did not show any DNA variations in the coding regions of the WDR36 gene. Without being held to theory, it is hypothesized that other mutations interfering with the mRNA splicing machinery, mutations within the promoter of this gene, or present of other DNA rearrangements such as insertion, deletion or duplication are responsible for the POAG phenotype in these 2 families. Alternatively, it is hypothesized that another POAG locus is located within the identified genetic linkage and, therefore, mutations in another gene are responsible for these 2 families.

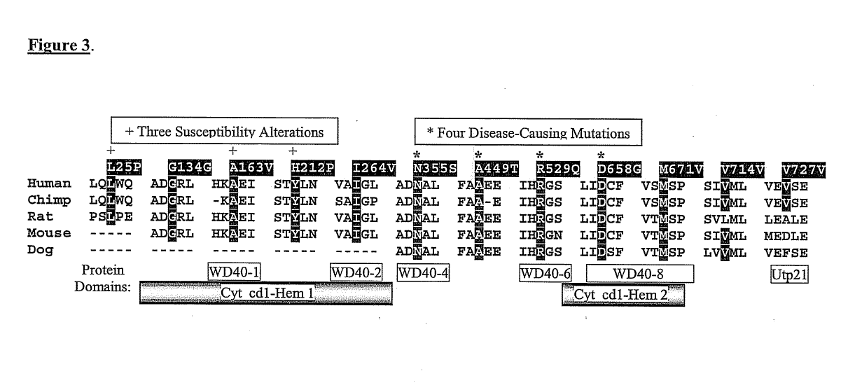

Mutation analysis of WDR36 in 130 unrelated affected individuals revealed a total of 24 allelic variants, of which 12 were amino acids coding and 12 involved intronic alterations (Table 2). Comparative sequence alignments of this gene between human, chimp, dog, rat and mouse revealed that 11 of these amino acids (except V727) have been fully conserved between these species (FIG. 3). Evolutionary conservation of the 4 identified disease-causing mutations (N355S, A449T, R529Q and D658G) in at least 5 different species and their absence in a minimum of 200 human normal control chromosomes imply that these 4 amino acids perform a fundamentally important role in the organization of WDR36 protein.

TABLE 2
Distribution of 24 Sequence Variations Identified in the WDR36 Gene

| | | | | | |
|---|---|---|---|---|---|
| Exon or | | Predicted | Protein | Observed Mutations/ | |
| Intron | cDNA | Protein | Domain | Total No. of POAG | Observed Mutations/ |
| Position | Change* | Change | Affected | Families (%) | Total No. of Normal Chromosomes (%) |

Predicted Disease-Causing Mutations

| | | | | | |
|---|---|---|---|---|---|
| EX-1 | c.91C > A | P31T | None | 1/171 (0.58) | N/A |
| EX-8 | c.1064A > G | N355S | WD40 Domain 4 | 1/171 (0.58) | 0/200 (0.00) |
| EX-11 | c.1345G > A | A449T | None | 2/171 (1.17) | 0/200 (0.00) |
| EX-13 | c.1514G > T | C505F | WD40 Domain 6 | 1/171 (0.58) | N/A |
| EX-13 | c.1586G > A | R529Q | WD40 Domain 6 | 1/171 (0.58) | 0/200 (0.00) |
| EX-14 | c.1642G > A | D548N | WD40 Domain 6 | 1/171 (0.58) | 0/100 (0.00) |
| EX-17 | c.1973A > G | D658G | WD40 Domain 8 | 13/711 (1.83)⁺ | 0/476 (0.00) |
| | | | Cyt_cd1-Hem 2 | | |

| | | |
|---|---|---|
| Sum of All Predicted Disease-Causing Mutations: | (5.90)^{ψ} | (0.00) |

Potential Disease-Susceptibility Mutations

| | | | | | |
|---|---|---|---|---|---|
| EX-1 | c.74T > C | L25P | None | 5/171 (2.92) | 2/428 (0.47) |
| EX-4 | c.488C > T | A163V | WD40 Domain 1 | 4/171 (2.34) | 1/422 (0.24) |
| | | | Cyt_cd1-Hem 1 | | |
| EX-5 | c.646-647TA > CC | H212P | Cyt_cd1-Hem 1 | 6/171 (3.51) | 3/200 (1.50) |

| | | |
|---|---|---|
| Sum of All Potential Disease-Susceptibility Mutations: | (8.77) | (2.21) |

Amino Acid Polymorphisms

| | | | | | |
|---|---|---|---|---|---|
| EX-3 | c.402C > T | G134G | Cyt_cd1-Hem 1 | 6/171 (3.51) | 100% Linkage Disequilibrium |
| | | | | | with H212P |
| EX-7 | c.790A > G | I264V | WD40 Domain 2 | 91/171 (53.22) | (SNP#: rs11241095) |
| | | | Cyt_cd1-Hem 1 | | |
| Ex-17 | c.2011A > G | M671V | WD40 Domain 8 | 3/171 (1.75) | (SNP#: rs11956837) |
| | | | Cyt_cd1-Hem 2 | | |
| EX-18 | c.2142C > G | V714V | None | 28/171 (16.37) | 95% Linkage Disequilibrium with |
| | | | | | IVS18 + 216C > T; (SNP#: rs17624563) |
| EX-19 | c.2181A > T | V727V | Utp21 | 100/171 (58.48) | (SNP#: rs13186912) |

Intronic Polymorphisms

| | | | | |
|---|---|---|---|---|
| IVS3 | IVS3 − 113G > A | N/A | 31/171 (18.13) | (SNP#: rs13153937) |
| IVS4 | IVS4 − 27A > G | N/A | 1/171 (0.58) | Present in Normal Population |
| IVS4 | IVS4 − 139A > T | N/A | 1/171 (0.58) | Present in Normal Population |
| IVS5 | IVS5 + 30C > T | N/A | 36/171 (21.05) | (SNP#: rs10038177) |
| IVS7 | IVS7 + 105A > G | N/A | 2/171 (1.17) | Present in Normal Population |
| IVS7 | IVS7 − 39T > G | N/A | 2/171 (1.17) | 0/90 (0.00) |
| IVS8 | IVS8 + 92G > A | N/A | 1/171 (0.58) | 0/90 (0.00) |
| IVS12 | IVS12 + 90C > T | N/A | 122/171 (71.34) | (SNP#: rs10043631) |
| IVS13 | IVS13 + 89G > A | N/A | 51/171 (29.82) | Present in Normal Population |
| IVS14 | IVS14 + 89C > A | N/A | 108/171 (63.16) | (SNP#: rs13161853) |
| IVS16 | IVS16 − 30A > G | N/A | 87/171 (50.88) | Present in Normal Population |
| IVS18 | IVS18 − 83A > G | N/A | 1/171 (0.58) | N/A |
| IVS18 | IVS18 + 217C > T | N/A | 27/171 (15.79) | Present in Normal Population |
| IVS21 | IVS21 + 60G > C | N/A | 13/171 (7.60) | (SNP#: rs2290680) |
| IVS22 | IVS22 + 129G > A | N/A | 11/171 (6.43) | (SNP#: rs11951907) |
| IVS22 | IVS22 + 189T > C | N/A | 9/171 (5.26) | (SNP#: rs11955335) |
| IVS22 | IVS22 − 202A > G | N/A | 22/171 (12.86) | (SNP#: rs4530809) |
| 3′UTR | 3′UTR + 45C > G | N/A | 1/171 (0.58) | 9/100 (9.00) |

*Based on GenBank Accession #: NM_139281
⁺Five D658G mutations identified in the same group of 171 POAG families (2.92%) plus another 8 mutations in additional 540 unrelated subjects (i.e., 5 out of 268 familial and 3 out of 272 sporadic cases) that were tested only for this one particular mutation. Altogether, 13 D658G mutations were observed in 711 (2.1%) unrelated familial and sporadic cases of POAG.
^{ψ}This figure is 7.02% (12 out of 171) for the same 171 POAG families that were fully sequenced for WDR36.

Although one of the WDR36 disease-causing mutations identified in this study (A449T) is not part of a known protein motif, the other three are located in separate G-beta WD40 repeats (FIG. 3). The N355S mutation in exon 8 maps to the 4^{th} WD40 repeat (covering a.a. 321-361), the R529Q in exon 13 maps to the 6^{th} WD40 repeat (a.a. 525-567) and D658G in exon 17 maps to the 8^{th} WD40 repeat (a.a. 653-692). The G proteins are a family of membrane-associated proteins that act as intermediaries in transduction of the signals generated by transmembrane receptors. The G-beta sunbit is required for membrane anchoring and receptor recognition. Structurally, the G-beta subunit includes eight tandem repeats of about 40 residues, each containing a central Trp-Asp dipeptide (thus named WD-40 repeats). Therefore, mutations affecting the structure of these WD-40 repeats may interfere with interaction of WDR36 with other proteins. This repetitive WD-40 segment is also present in over 250 proteins that are encoded by a variety of genes mapping to every one of the human chromosomes. None of the WDR36 detected amino acid alterations in this study were located in the AMP-binding domain (a.a. 553-564) and only one Single Nucleotide Polymorphism or SNP (V1727V) was identified from within the Utp21 domain (a.a. 724-948).

The D658G mutation (FIG. 2d and FIG. 3) that is located within the ₈th WD40 repeat, also maps to C-terminal part of the Cytochrome heme cd1 (cyt cd1) domain (a.a. 109-318 and 537-686). This “cyt cd1” is part of a bi-functional enzyme with cytochrome oxidase activity. It is interesting another member of the Cytochrome P450 family (CYPlBl) has been identified as involved in the etiology of primary congenital glaucoma. Therefore, it is hypothesized that there is a functional association between WDR36 and CYPlBl, as mutations in these two genes are responsible for two different forms of glaucoma.

Since D658G mutation was originally observed in 5 out of 130 unrelated and familial POAG subjects, the presence of this one particular mutation in an additional 540 unrelated affected individuals from different POAG subgroups was further tested. This mutation creates a new recognition site (Bgl I) thus providing a rapid method for its screening (FIG. 2e). In total, 13 out of 670 (1.94%) subjects tested were found to be heterozygous for D658G (7 with high- and 6 with low-pressure glaucoma). Originally, the entire of WDR36 gene was sequenced in a total of 130 familial POAG cases and 3 mutations of N355S, A449T and R529Q in 4 families (3.08%) and the common mutation of D658G in 5 families were identified. Altogether, 9 out of 130 (6.92%) families that were fully sequenced showed mutations in the WDR36 gene. When additional 540 unrelated affected subjects (268 familial and 272 sporadic) were only tested for presence of D658G, this mutation was observed in another 5 familial and 3 sporadic cases. Altogether, D658G was observed in 2.51% of familial (10 out of 398) and 1.10% of sporadic (3 out of 272) cases thus providing a combined frequency of 1.94% (13 out of 670) for this one mutation alone. When this is added to the frequency of 3.08% obtained from the other 3 mutations, a minimum mutation rate of 5.02% is obtained for this gene. After the entire of WDR36 gene in the above-mentioned 540 familial and sporadic cases is fully sequenced, it is anticipated that additional mutations will be identified thus, altering the mutation frequency of 5.02%-6.92% as presented herein. In summary,4 different WDR36 mutations in 17 subjects with either high-pressure (65% of subjects) or low-pressure (35% of subjects) primary open angle glaucoma were identified. This observation indicates that the WDR36 gene is involved in etiology of both types of glaucoma and, IOP as traditionally used to group these into two separate clinical entities, may not be supported by this study and by recent molecular delineation of this group of optic neuropathies.

Three other amino acid alterations of L25P, A163V and H212P in a group of 15 POAG subjects were also identified Table 2). As these 3 alterations were absent in all of the public databases, their presence was screened in 428, 422 and 200 normal control chromosomes, respectively. As the ratio of their presence in the POAG group was significantly higher than that obtained for the normal group (11.55% vs. 2.21%, respectively), these alterations are identified as potential disease susceptibility alterations (Table 2, FIG. 3).

Three amino acid polymorphisms were also observed in POAG or normal control subjects (1264V, M671V and V727V). In addition to these, the Ensemble database lists 2 other polymorphisms (S901 and A149A) that were not observed in our study. The 2 other silent amino acid polymorphisms (G134G, and V714V) identified in this study were also present in the public EST databases. It is interesting that during this study (Table 2) 100% linkage disequilibrium between G134G and H212P, and 95% between V714V and IVS 18+216C>T were observed. Of the 12 intronic alterations that were identified in this study, only 4 have previously been deposited in the SNP databases (Table 2). Therefore, a total of 90 normal control chromosomes were screened for the presence of these previously unreported intronic alterations. As shown in table 2, only two of these changes (i.e., IVS7−39T>G and IVS8+92G>A) were absent in the normal control chromosomes.

### Example 3

### Expression Study of WDR36 at RNA Level RNA Analysis

Under a dissecting microscope, the lens, iris, ciliary body, ciliary muscle, trabecular meshwork, sclera, retina, and optic nerve were micro-dissected and immediately used for RNA extraction. The TRIzol® reagent method (Invitrogen) was used to isolate RNA from these ocular tissues. For RT-PCR, cDNA were made from purified RNAs, using random hexamer primers and Superscript™ III reverse transcriptase (Invitrogen). Furthermore, expression of the WDR36 gene in human and mouse tissues were investigated by Northern hybridization. Radiolabeled probes were prepared from a 691-bp and 693-bp PCR fragment amplified from human and mouse cDNA respectively. Expression of this gene was studied by use of the same probe and a premade blot (Clontech) containing poly(A)+mRNA (2 μg/lane) from heart, brain, placenta, lung, liver, skeletal muscle, kidney and pancreas in human, and heart, brain, spleen, lung, liver, skeletal muscle, kidney and testis in mouse.

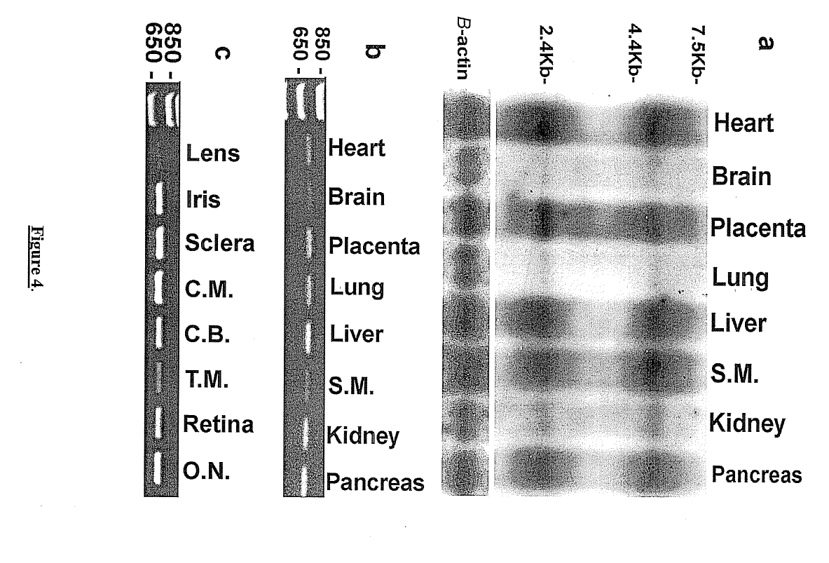

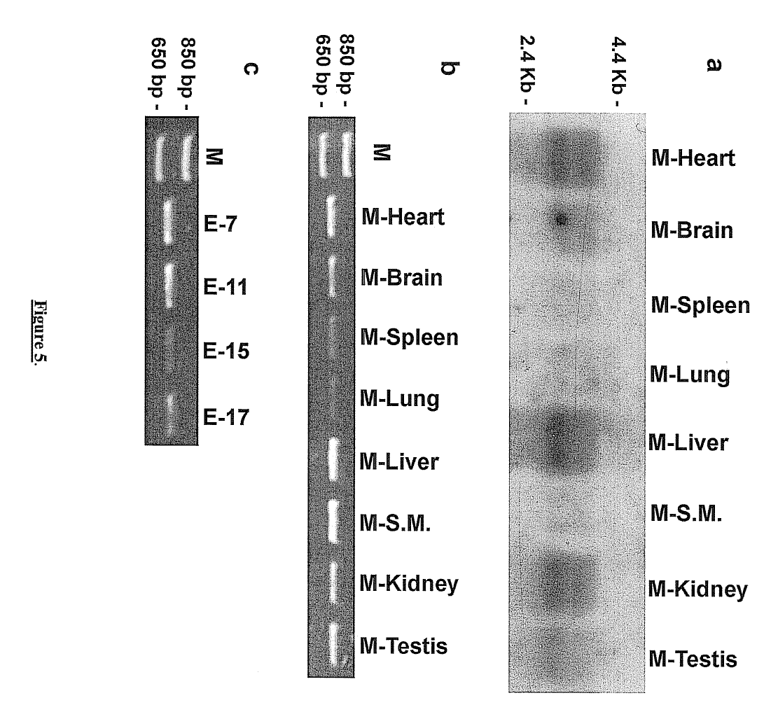

As a first step in determining the role of WDR36 in glaucoma pathogenesis, expression of this gene in different human ocular and non-ocular tissues by Northern blot and Reverse Transcription-Polymerase Chain Reaction (RT-PCR) was studied. Northern blotting of several human non-ocular mRNA samples revealed two different transcripts (i.e., 5.9-kb and 2.5-kb) that are highly expressed in heart, placenta, liver, skeletal muscle and pancreas (FIG. 4a). Heart, placenta, liver, smooth muscle and pancreas exhibited the strongest signals. By RT-PCR, similar WDR36 expressions were also detected in heart, brain, placenta, lung, liver, skeletal muscle, kidney, and pancreas (FIG. 4b). Placenta, lung, liver, kidney and pancreas produced the strongest signals. WDR36 expression was also detected in a number of ocular tissues including lens, iris, sclera, ciliary muscles, ciliary body, trabecular meshwork, retina, and optic nerve (FIG. 4c). Strong gene expression was observed in iris, sclera, ciliary muscle, ciliary body, retina and optic nerve. As a comparative study, we also determined mRNA expression patterns for the orthologue of this gene (Wdr36) in mouse. Northern blotting of Wdr36 in a panel of adult mouse tissues revealed analogous expression patterns to human with only one exception; in skeletal muscle the expression was stronger in human (FIG. 5a). The strongest signals were observed in heart, liver, kidney and testis. By RT-PCR, high levels of Wdr36 expression were detected in mouse heart, brain, liver, skeletal muscle, kidney, testis and lower expressions in spleen and lung (FIG. 5b). Similar to Northern blotting, spleen and lung had the weakest signals. Study of Wdr36 during various stages of mouse embryonic developments showed that this gene is detectable in 7-days old embryos (FIG. 5c).

By Northern analysis 2 different transcript sizes were detected in human and mouse (FIG. 4-5). However, it is not clear at this point whether these 2 transcripts were produced by alternative splicing or by use of 2 different promoters. For human, intense hybridization was observed in heart, placenta, liver, skeletal muscle, kidney and pancreas (FIG. 4a-b). In mouse, heart, liver and kidney produced the highest signal (FIG. 5a-b). By RT-PCR we determined expression of WDR36 in lens, iris, sclera, ciliary muscles, ciliary body, trabecular meshwork, retina, and optic nerve (FIG. 4c). In mouse, expressions were detected in 7-, 11-, 15- and 17-days old developing embryos (FIG. 5c).

Although the in-vivo function of WDR36 is yet unknown, identification of this gene as an adult-onset POAG gene provides an opportunity to find and to study biochemical pathways that are expected to be involved in the pathogenesis of this group of optic neuropathies. WDR36 contains multiple G-beta WD40 repeats that are also present in a large family of proteins with diverse functions. Since this WD40 repeat motif is also involved in protein-protein interaction and as WDR36 is now implicated in the etiology of POAG, this finding provides an opportunity to search for WDR36-interacting proteins aiming to identify other proteins/genes that have direct functional effect on the pathophysiology of this blinding condition. Additionally, since different mutations in WDR36 were observed in both high- and low-pressure glaucoma, molecular screening of this gene may provide a useful tool for presymptomatic detection of individuals at risk in families or in the elderly population.

The teachings of the references cited herein are incorporated by reference in their entirety.

While this invention has been particularly shown and described with references to preferred embodiments thereof, it will be understood by those skilled in the art that various changes in form and details may be made therein without departing from the spirit and scope of the invention as defined by the appended claims.

What is claimed is:

1. An isolated nucleic acid molecule comprising a portion of a GLC1G locus on chromosome band 5q22.1, wherein the nucleic acid has an alteration in a nucleotide sequence, and wherein the alteration is a glaucoma-causing alteration or a glaucoma-susceptibility alteration.

2. The isolated nucleic acid of claim 1, wherein the alteration is located in the WDR36 gene.

3. The isolated nucleic acid of claim 2, wherein the alteration is located in a nucleic acid sequence coding for a WD40 domain of the WDR polypeptide.

4. The isolated nucleic acid of claim 2, wherein the WDR36 gene comprises SEQ ID NO: 1, and wherein the alteration is a change of 91C>A in the WDR36 gene, corresponding to an P to T change at codon 31 (P31T) of the WDR36 protein; a change of 1064A>G in the WDR36 gene, corresponding to an N to S change at codon 355 (N355S) of the WDR36 protein; a change of 1345G>A in the WDR36 gene, corresponding to an A to T change at codon 449 (A449T) of the WDR36 protein; a change of 1514G>T in the WDR36 gene, corresponding to a C to F change at codon 505 (C505F); a change of 1586G>A in the WDR36 gene, corresponding to an R to Q change at codon 529 (R529Q) of the WDR36 protein; a change of 1642G>A in the WDR36 gene, corresponding to a D to N change at codon 548 (D548N); a change of 1973A>G in the WDR36 gene, corresponding to an D to G change at codon 658 (D658G) of the WDR36 protein; a change of 74T>C in the WDR36 gene, corresponding to an L to P change at codon 25 (L25P) of the WDR36 protein; a change of 488C>T in the WDR36 gene, corresponding to an A to V change at codon 163 (A163V) of the WDR36 protein; a change of 646-647TA>CC in the WDR36 gene, corresponding to a H to P change at codon 212 (H212P) of the WDR36 protein; or a combination comprising one or more of the foregoing alterations.

5. The isolated nucleic acid of claim 1 wherein the glaucoma is an open-angle glaucoma.

6. The isolated nucleic acid of claim 1 wherein the glaucoma is adult-onset primary open-angle glaucoma.

7. The isolated nucleic acid of claim 2, comprising an allele-specific oligonucleotide comprising 10 to 100 contiguous nucleotides of the WDR36 gene, wherein the allele-specific oligonucleotide comprises the glaucoma-causing alteration or glaucoma-susceptibility alteration.

8. The isolated nucleic acid of claim 7, wherein the allele-specific oligonucleotide comprises 15 to 60 contiguous nucleotides of the WDR36 gene.

9. The isolated nucleic acid of claim 7, wherein the WDR36 gene comprises SEQ ID NO: 1, and wherein the alteration is a change of 91C>A in the WDR36 gene, corresponding to an P to T change at codon 31 (P31 T) of the WDR36 protein; a change of 1064A>G in the WDR36 gene, corresponding to an N to S change at codon 355 (N355S) of the WDR36 protein; a change of 1345G>A in the WDR36 gene, corresponding to an A to T change at codon 449 (A449T) of the WDR36 protein; a change of 1514G>T in the WDR36 gene, corresponding to a C to F change at codon 505 (C505F); a change of 1586G>A in the WDR36 gene, corresponding to an R to Q change at codon 529 (R529Q) of the WDR36 protein; a change of 1642G>A in the WDR36 gene, corresponding to a D to N change at codon 548 (D548N); a change of 1973A>G in the WDR36 gene, corresponding to an D to G change at codon 658 (D658G) of the WDR36 protein; a change of 74T>C in the WDR36 gene, corresponding to an L to P change at codon 25 (L25P) of the WDR36 protein; a change of 488C>T in the WDR36 gene, corresponding to an A to V change at codon 163 (A163V) of the WDR36 protein; a change of 646-647TA>CC in the WDR36 gene, corresponding to a H to P change at codon 212 (H212P) of the WDR36 protein; or a combination comprising one or more of the foregoing alterations.

10. The isolated nucleic acid of claim 7, wherein the allele-specific oligonucleotide is in the form of an array attached to a solid support.

11. An isolated or purified polypeptide encoded by the altered nucleic acid of claim 1.

12. A method for detecting the presence or absence of a WDR36-associated glaucoma or of a WDR36-associated greater than normal risk of glaucoma in an individual comprising:
assessing a sample from the individual for an alteration in a WDR36-associated nucleic acid, or
assessing the sample for an alteration in a WDR36-associated polypeptide,
wherein the alteration in the nucleic acid or in the polypeptide is a glaucoma-causing alteration or a glaucoma-susceptibility alteration.

13. The method of claim 12, further comprising obtaining the sample from the individual.

14. The method of claim 12, wherein assessing is from a nucleic acid sample, and wherein the WDR36 gene comprises SEQ ID NO: 1, and wherein the alteration is a change of 91C>A in the WDR36 gene, corresponding to an P to T change at codon 31 (P31T) of the WDR36 protein; a change of 1064A>G in the WDR36 gene, corresponding to an N to S change at codon 355 (N355S) of the WDR36 protein; a change of 1345G>A in the WDR36 gene, corresponding to an A to T change at codon 449 (A449T) of the WDR36 protein; a change of 1514G>T in the WDR36 gene, corresponding to a C to F change at codon 505 (C505F); a change of 1586G>A in the WDR36 gene, corresponding to an R to Q change at codon 529 (R529Q) of the WDR36 protein; a change of 1642G>A in the WDR36 gene, corresponding to a D to N change at codon 548 (D548N); a change of 1973A>G in the WDR36 gene, corresponding to an D to G change at codon 658 (D658G) of the WDR36 protein; a change of 74T>C in the WDR36 gene, corresponding to an L to P change at codon 25 (L25P) of the WDR36 protein; a change of 488C>T in the WDR36 gene, corresponding to an A to V change at codon 163 (Al 63V) of the WDR36 protein; a change of 646-647TA>CC in the WDR36 gene, corresponding to a H to P change at codon 212 (H212P) of the WDR36 protein; or a combination comprising one or more of the foregoing alterations.

15. The method of claim 12, wherein the glaucoma is an open-angle glaucoma.

16. The method of claim 12, wherein the glaucoma is adult-onset primary open-angle glaucoma.

17. The method of claim 12, wherein assessing comprises utilizing an array of nucleic acid molecules attached to a solid support.

18. The method of claim 12, wherein assessing comprises sequencing at least a portion of a WDR36 nucleic acid, or hybridizing a nucleic acid probe to a WDR36 nucleic acid.

19. The method of claim 12, wherein the individual has an elevated intraocular pressure.

20. The method of claim 12, wherein the individual has a normal intraocular pressure.
